(19) United States
(12) Patent Application Publication
(10) Pub. No.: US 2022/0033808 A1
(43) Pub. Date: Feb. 3, 2022

(54) METHODS AND COMPOSITIONS FOR NUCLEIC ACID-GUIDED NUCLEASE CELL TARGETING SCREEN

(71) Applicant: Spotlight Therapeutics, Hayward, CA (US)

(72) Inventors: Akshay Tambe, Oakland, CA (US); Hariharan Jayaram, Menlo Park, CA (US); Steven Strutt, San Francisco, CA (US)

(73) Assignee: Spotlight Therapeutics, Hayward, CA (US)

(21) Appl. No.: 17/507,324

(22) Filed: Oct. 21, 2021

Related U.S. Application Data
(63) Continuation of application No. PCT/US2020/029864, filed on Apr. 24, 2020.
(60) Provisional application No. 62/838,053, filed on Apr. 24, 2019.

(51) Int. Cl. C12N 15/10; C12N 15/11; C12N 15/82; C12N 15/90; C12N 9/22

(52) U.S. Cl. CPC C12N 15/1065; C12N 15/11; C12N 15/8213; C12N 15/905; C12N 2310/20; C12N 15/1082; C12N 2800/80; C07K 2319/09; C07K 2319/10; C12N 9/22

(57) ABSTRACT

Methods and compositions related to a nucleic acid-guided nuclease cell targeting screen are provided. The invention relates to compositions and methods for identifying cell targeting proteins that, when associated with a nucleic acid-guided nuclease (such as Cas9), enables at least the nucleic acid-guided nuclease to be targeted to the surface of a target cell or internalized by a target cell, i.e., a cell targeted by the cell targeting agent.

### BACKGROUND OF THE INVENTION

CRISPR-associated RNA-guided endonucleases, such as Cas9, have become a versatile tool for genome engineering in various cell types and organisms (see, e.g., U.S. Pat. No. 8,697,359). Guided by a guide RNA, such as a dual-RNA complex or a chimeric single-guide RNA, RNA-guided endonucleases (e.g., Cas9) can generate site-specific double-stranded breaks (DSBs) or single-stranded breaks (SSBs) within target nucleic acids (e.g., double-stranded DNA (dsDNA), single-stranded DNA (ssDNA), or RNA). When cleavage of a target nucleic acid occurs within a cell (e.g., a eukaryotic cell), the break in the target nucleic acid can be repaired by nonhomologous end joining (NHEJ) or homology directed repair (HDR). In addition, catalytically inactive RNA-guided endonucleases (e.g., Cas9) alone or fused to transcriptional activator or repressor domains can be used to alter transcription levels at sites within target nucleic acids by binding to the target site without cleavage. However, the ability to target RNA-guided endonucleases to specific cells or tissues remains a challenge. There is thus an unmet need for identifying RNA-guided endonucleases with the capability of targeting desired cells or tissues.

### SUMMARY OF THE INVENTION

Provided herein are methods and compositions relating to a screen for identifying cell targeting agents capable of targeting a nucleic-acid guided nuclease to a cell and/or promoting internalization of the nucleic acid-guided nuclease into the cell, or a compartment thereof (e.g., the nucleus).

In one aspect, provided herein is a method of identifying a cell targeting agent, the method comprising providing a plurality of ribonucleoproteins (RNPs) each comprising an RNA-guided nuclease fusion protein and a unique identifying RNA (uiRNA), wherein the RNA-guided nuclease fusion protein comprises an RNA-guided nuclease, or a functional fragment thereof, and a test protein; and wherein the uiRNA comprises a guide RNA (gRNA) and a sequence identifier; contacting the RNPs with a population of target cells; isolating RNA from the population of target cells, thereby obtaining isolated RNA; and testing the isolated RNA for the presence of the identifier sequence, wherein the presence of the identifier sequence indicates that the test protein is a cell targeting agent.

In another aspect, provided herein is a method of identifying a cell targeting agent, the method comprising: providing a vector encoding an RNA-guided nuclease fusion protein comprising an RNA-guided nuclease, or a functional fragment thereof, and a test protein, and encoding a unique identifying RNA (uiRNA) comprising a guide RNA (gRNA) and a sequence identifier; transferring the vector to a host cell suitable to express the RNA-guided nuclease fusion protein and the uiRNA; expressing the RNA-guided nuclease fusion protein and the uiRNA in the host cell, such that ribonucleoproteins (RNPs) each comprising the RNA-guided nuclease fusion protein and the uiRNA are formed; isolating the RNPs from the host cell; contacting the RNPs with a population of target cells; isolating RNA from the population of target cells; and testing the isolated RNA for the presence of the identifier sequence, wherein the presence of the identifier sequence indicates that the test protein is a cell targeting agent.

In some embodiments, portions of the vector encoding the nucleic acid sequence identifier and the test protein are sequenced prior to the vector being transferred into the host cell, thereby providing a reference for identifying the test protein.

In some embodiments, the presence of the identifier sequence is detected using polymerase chain reaction (PCR) or a nucleic acid microarray.

In some embodiments, the vector is in a plurality of vectors and the plurality of vectors are transferred into host cells under conditions such that the average vector per host cell is 1 or more. In some embodiments, the vector is in a plurality of vectors and the plurality of vectors are transferred into host cells under conditions such that the average vector per host cell is less than 1

In some embodiments, the vector comprises a first promoter operatively linked to a nucleic acid sequence encoding the RNA-guided nuclease fusion protein, and comprises a second promoter operatively linked to a nucleic acid sequence encoding the uiRNA. In certain embodiments, the first and second promoter are each inducible such that the expression level of the RNA-guided nuclease fusion protein and the expression level of the uiRNA can be controlled to obtain RNPs. In certain embodiments, the first and/or second promoter is T7 or T5.

In some embodiments, the first and/or second promoter is a constitutive promoter.

In some embodiments, the vector comprises a selectable marker to select for the host cell into which the vector has been transferred. In some embodiments, the selectable marker is a gene that upon expression confers resistance to a selection agent (e.g., a drug, e.g., antibiotic). In some embodiments, the selectable marker is a gene that upon expression confers an identifiable phenotype. For example, the selectable marker may be a fluorescent marker that confers fluorescence in cells carrying the vector that can be identified visually or by machine, e.g., flow cytometry.

In some embodiments, the vector comprises a bacterial origin of replication.

In some embodiments, the vector comprises a eukaryotic origin of replication.

In some embodiments, the cell targeting agent either internalizes into a compartment of the target cell or binds to the cell surface of the target cell. In certain embodiments, the compartment is a membrane-bound organelle or cytoplasm. In certain embodiments, the membrane-bound organelle is a nucleus, endoplasmic reticulum, Golgi apparatus, vacuole, lysosome, endosome, or mitochondria.

In some embodiments, the isolated RNA is obtained from membrane-bound organelles that are extracted from the target cell prior to RNA isolation. In certain embodiments, the membrane-bound organelle is a nucleus, endoplasmic reticulum, Golgi apparatus, vacuole, lysosome, endosome, or mitochondria.

In some embodiments, the isolated RNA is obtained from cytoplasm that is extracted from the target cell prior to RNA isolation.

In some embodiments, the testing step comprises reverse-transcribing the isolated RNA to producing cDNA, and sequencing the cDNA to determine the presence of the identifier sequence.

In some embodiments, the testing step comprises sequencing the isolated RNA to determine the presence of the identifier sequence.

In some embodiments, the test protein is a peptide.

In some embodiments, the test protein is an antigen-binding protein.

In some embodiments, the antigen binding protein is a nanobody, a domain antibody, an scFv, a Fab, a diabody, a BiTE, a diabody, a DART, a minibody, a F(ab′)₂, an intrabody, or an antibody mimetic. In certain embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, or a versabody, an aptamer, or a cyclotide.

In some embodiments, the test protein is a ligand, or portion thereof.

In some embodiments, the host cell is a eukaryotic cell.

In some embodiments, the host cell is a bacterial cell. In certain embodiments, the bacterial cell is E. coli.

In some embodiments, the RNA-guided nuclease is a Class 2 Cas polypeptide. In certain embodiments, the Class 2 Cas polypeptide is a Type II, Type V, or Type VI Cas polypeptide. In certain embodiments, the Type II Cas polypeptide is Cas9.

In some embodiments, the target cells are mammalian cells. In certain embodiments, the mammalian cells are hematopoietic stem cells (HSC), neutrophils, T cells, B cells, dendritic cells, macrophages, ocular cells, or fibroblasts.

In another aspect, provided herein is a cell expression vector comprising: a nucleic acid encoding an RNA-guided nuclease operably linked to a cloning site for inserting a nucleic acid of a test protein, thereby forming an RNA-guided nuclease fusion protein comprising the RNA-guided nuclease and the test protein; and a nucleic acid encoding a unique identifying RNA (uiRNA), wherein the uiRNA comprises a guide RNA and a sequence identifier.

In some embodiments, expression vector further comprises the nucleic acid encoding the test protein.

In some embodiments, the expression vector is a plasmid.

In some embodiments, the cell expression vector comprises a first promoter operatively linked to the nucleic acid sequence encoding the RNA-guided nuclease, and comprises a second promoter operatively linked to the nucleic acid sequence encoding the uiRNA. In certain embodiments, the first and second promoter each comprise an inducible element such that the expression level of the RNA-guided nuclease fusion protein and the expression level of the uiRNA can be controlled. In certain embodiments, the first and/or second promoter is T7 or T5.

In some embodiments, the first and/or second promoter is a constitutive promoter.

In some embodiments, the vector comprises a selectable marker. In some embodiments, the selectable marker is a gene that upon expression confers resistance to a selection agent (e.g., a drug, e.g., antibiotic). In some embodiments, the selectable marker is a gene that upon expression confers an identifiable phenotype. For example, the selectable marker may be a fluorescent marker that confers fluorescence in cells carrying the vector that can be identified visually or by machine, e.g., flow cytometry.

In some embodiments, the vector comprises a bacterial origin of replication.

In some embodiments, the vector comprises a eukaryotic origin of replication.

In some embodiments, the RNA-guided nuclease is a Class 2 Cas polypeptide. In certain embodiments, the Class 2 Cas polypeptide is a Type II, Type V, or Type VI Cas polypeptide. In certain embodiments, the Type II Cas polypeptide is Cas9.

In another aspect, provided herein is a kit comprising any of the cell expression vectors of the invention.

In some embodiments, the kit further comprises reagents for inserting the polynucleotide encoding the test protein into the cloning site of the cell expression vector.

In another aspect, provided herein is an isolated cell comprising an of the cell expression vectors of the invention. In certain embodiments, the cell is a eukaryotic cell or a bacterial cell. In some embodiments, the eukaryotic cell is a mammalian cell, an insect cell, or a yeast cell. In certain embodiments, the mammalian cell is a COP cell, an L cell, a C127 cell, an Sp2/0 cell, an NS-0 cell, an NIH3T3 cell, a PC12 cell, a PC12h cell, a BHK cell, a CHO cell, a COS1 cell, a COS3 cell, a COST cell, a CV1 cell, a Vero cell, a HeLa cell, an HEK-293 cell, a PER C6 cell, a cell derived from diploid fibroblasts, a myeloma cell, or HepG2. In certain embodiments, the yeast cell is Pichia pastoris or Saccharomyces cerevisiae. In certain embodiments, the bacterial cell is an E. coli cell. In certain embodiments, the insect cell is a Spodoptera frugiperda cell.

In another aspect, provided herein is a method for producing at least one RNP comprising the RNA-guided nuclease fusion protein and the uiRNA comprising culturing a cell comprising any of the expression vectors of the invention in a cell culture medium under conditions allowing expression and assembly of the at least one RNP. In some embodiments, at least one RNP is/are secreted into the cell culture medium and the method further comprises the step of isolating from the cell culture medium the at least one RNP.

In another aspect, provided herein is a library of cell expression vectors comprising a plurality of any of the cell expression vectors of the invention. In some embodiments, each of the cell expression vectors comprises a different sequence identifier.

In further embodiments, provided herein is a method of producing a sublibrary of variants of a selected test agent, and testing the sublibrary to identify variants with the desired activity following contacting the sublibrary with a target cell population, using the methods set forth herein.

### BRIEF DESCRIPTION OF THE DRAWINGS

FIG. 1 graphically depicts a flowchart outlining the steps in an exemplary nucleic acid-guided nuclease cell targeting screen for cell penetrating peptides that can effectively facilitate internalization of Cas9.

FIGS. 2A-2C show results of a high-throughput cloning process to prepare a library of CPP-Cas9 vectors each encoding a unique identifying RNA (uiRNA) associated with a test CPP. FIG. 2A depicts a map of a nucleic acid encoding a uiRNA and 6×His-CPP*-Cas9-2×NLS (“6×His” disclosed as SEQ ID NO: 22) (the asterisk indicates that the CPP is variable). FIG. 2B shows a photograph of an exemplary agar plate containing colonies from a small library of approximately 5000 E. coli transformants. FIG. 2C shows the results of a gel electrophoresis analysis of two replicates of PCR amplified CPP-Cas9 plasmid libraries (˜1200 bp band; lanes 2 and 3) as compared to a nucleic acid ladder (lane 1).

FIGS. 3A and 3B show results related to the sequencing of a CPP-Cas9 plasmid library. FIG. 3A graphically depicts results comparing the plasmid-seq unique molecular identifier (UMI) counts between two library replicates. FIG. 3B graphically depicts the library coverage distribution for each CPP-Cas9-fusion represented in the library sorted by abundance on the x-axis and with relative abundance (counts per million) indicated on the y-axis.

FIGS. 4A-4B shows the results of studies to assess plasmid non-uniformity. FIG. 4A graphically depicts the number of plasmid UMIs per CPP-Cas9 fusion for two library replicates, which is indicative of library bias or cloning bias in E. coli (e.g., copy number or growth rate). FIG. 4B graphically depicts the number of sgRNA barcodes (i.e., uiRNA) per CPP-Cas9 fusion, which is indicative of library assembly bias. FIG. 4C graphically depicts the number of UMIs per sgRNA barcodes (i.e., uiRNA), which is indicative of sequencing bias.

FIGS. 5A-5D show results related to the co-purification of the library of RNPs formed between CPP-Cas9 fusions and barcoded or GFP sgRNAs expressed from plasmids in the plasmid library in E. coli. FIG. 5A shows an image of an SDS-PAGE gel analysis (coomasie stained) of protein (e.g., Cas9, 150 kDa band) in samples collected from each indicated RNP purification step. FIG. 5B shows an image of a gel electrophoresis analysis (2% agarose; SyBr safe dye) of nucleic acids in samples collected from each indicated RNP purification step. FIG. 5C graphically depicts a chromatogram from size exclusion analysis of the purified RNPs on a S200 column. FIG. 5D shows an image of a gel electrophoresis analysis (2% agarose, SyBr Safe dye) of bulk RNAs extracted from the purified RNPs. Synthego sgRNA is shown as a positive control.

FIG. 6 shows an image of a gel electrophoresis analysis (2% agarose gel, SyBr Safe dye) of products obtained from reverse-transcription of RNAs that co-purified with the library of CPP-Cas9 RNPs, with Barcoded or GFP sgRNA products, a no template negative control, and a Synthego sgRNA positive control shown.

FIG. 7 shows an image of a gel electrophoresis analysis (2% agarose gel, SyBr Safe dye) of samples from a DNA cleavage assay, in which a library of CPP-Cas9 RNPs having target sgRNA (GFP) and nontarget sgRNA (barcode) were incubated with dsRNA. Bands corresponding to uncleaved and cleaved dsRNA are indicated. dsRNA from a no RNP control condition is also shown.

FIGS. 8A and 8B graphically depicts results from a RNA-seq analysis of RNAs co-purified with the library of CPP-Cas9 RNPs, comparing inter-replicate RNA-seq UMI counts (FIG. 8A) and sample correlation for plasmid vs RNP abundance (FIG. 88).

FIG. 9 shows an image of a gel electrophoresis analysis of nuclear RNAs isolated from human or mouse T cells co-incubated with a library of CPP-Cas9 RNPs for either 1 hour or 5 hours. gRNAs are represented by the upper band. RNA from RNPs alone or a negative control (T cell or Human T cells co-incubated with buffer but no Cas9 RNP for 5 hours) were also assessed.

FIGS. 10A and 10B graphically depicts RNA-seq results comparing inter-replicate RNA-seq UMI counts for RNA isolated from stimulated human T cells incubated with the library of purified CPP-Cas9 RNPs for 1 hour (FIG. 10A) or 5 hours (FIG. 10B).

FIGS. 11A-11C graphically depicts results analyzing RNAs associated with differentially expressed and internalized CPP-Cas9 RNPs in human stimulated T cells co-incubated with the library of CPP-Cas9 RNPs for either 1 hour (FIG. 11A) or 5 hours (FIGS. 11B and 11C). The graphs compare the fold change of RNAs sequenced in the nuclear extractions (ATSeq-01C) obtained from the human stimulated T cells relative to RNAs sequenced in the starting material (pooled RNPs prior to co-incubation; ATSeq-01A) and plotted relative to total RNP abundance (ATSeq-01A; y-axis). FIG. 11C highlights key data points (see stars) representing RNAs associated with CPP-Cas9 RNPs that have a high abundance and high nuclear internalization in human stimulated T cells following 5 hours of co-incubation with the library of CPP-Cas9 RNPs. CPPs associated with the highlighted data points are summarized in Table 1.

FIGS. 12A-12D graphically depict results of a screen for CPP-Cas9 RNP internalization in fibroblasts. The fibroblasts were co-incubated with a pooled library of purified CPP-Cas9 RNPs for 1 hour at 37C, after which cells were washed and fractionated into nuclei and cytosol for further analysis by RNA-seq. FIG. 12A graphically depicts the results of a principal component analysis on the uiRNA counts from an input control, unfractionated cells, cytoplasmic fraction, and nuclear fraction. The results were further analyzed based on the co-incubation protocol used (low RNP concentration vs high RNP concentration protocol). FIG. 12B graphically depicts the fold change (x-axis) of RNAs in nuclear extractions relative to RNAs in an input control plotted in a volcano plot relative to P value (y-axis). RNAs associated with CPP-Cas9 RNPs displaying nuclear localization in fibroblasts following co-incubation with pooled CPP-Cas9 RNPs are shown in the upper right of portion of the graph (see boxed portion of FIG. 12B and starred hits in FIG. 12C). FIG. 12C highlights the top eight data points of FIG. 12B (see starred data points) representing RNAs associated with CPP-Cas9 RNPs having enriched nuclear internalization in fibroblasts. FIG. 12D graphically depicts the hydropathy (y-axis) and net charge per residue (x-axis) for CPPs identified in the screen. Each dot represents a peptide in FIGS. 12B and 12C, wherein the size of the dot indicates the P value (Log 10), and the shading indicates fold change (Log 10). The data on the bottom right of the graph indicate highly charged CPPs with a low degree of hydrophobicity. The circled data points in FIG. 12B and FIG. 12D correspond to data for the same CPP-Cas9 RNPs (e.g., circled data point 1 in each figure correspond to data for the same CPP-Cas9 RNP). Circled data point 1 corresponds to a highly charged CPP hit, and circled data point 2 corresponds to a nonpolar CPP hit.

### DETAILED DESCRIPTION OF THE INVENTION

The invention relates to compositions and methods for screening for cell targeting agents for targeting nucleic acid-guided gene editing polypeptides, such as Cas9, into a cell.

### I. Definitions

The term “nucleic acid-guided nuclease fusion protein” refers to a complex of molecules including a test agent conjugated to a nucleic acid-guided nuclease (e.g., a RNA-guided nuclease or a DNA-guided nuclease) that recognizes a nucleic acid sequence. An example of a nucleic acid-guided nuclease is a RNA-guided endonuclease, such as Cas9.

As used herein, a “nucleic acid-guided nuclease” refers to a protein that is targeted to a specific nucleic acid sequence or set of similar sequences of a polynucleotide chain via recognition of the particular sequence(s) by the modifying polypeptide itself or an associated molecule (e.g., RNA), wherein the polypeptide can modify the polynucleotide chain.

As used herein, the term “nucleic acid” refers to a molecule comprising nucleotides, including a polynucleotide, an oligonucleotide, or other DNA or RNA. In one embodiment, a nucleic acid is present in a cell and can be transmitted to progeny of the cell via cell division. In some instances, a nucleic acid is a gene (e.g., an endogenous gene) found within the genome of a cell within its chromosomes. In other instances, a nucleic acid is a mammalian expression vector that has been transfected into a cell. DNA that is incorporated into the genome of a cell using, e.g., transfection methods, is also considered within the scope of a “nucleic acid” as used herein, even if the incorporated DNA is not meant to be transmitted to progeny cells.

As used herein, the term “modifying a nucleic acid” refers to any modification to a nucleic acid targeted by a site-directed modifying polypeptide. Examples of such modifications include any changes to the amino acid sequence including, but not limited to, any insertion, deletion, or substitution of an amino acid residue in the nucleic acid sequence relative to a reference sequence (e.g., a wild-type or a native sequence). Such amino acid changes may, for example, may lead to a change in expression of a gene (e.g., an increase or decrease in expression) or replacement of a nucleic acid sequence. Modifications of nucleic acids can further include double stranded cleavage, single stranded cleavage, or binding of any RNA-guided endonuclease disclosed herein to a target site. Binding of a RNA-guided endonuclease can inhibit expression of the nucleic acid or can increase expression of any nucleic acid in operable linkage to the nucleic acid comprising the target site.

As used herein, the term “unique identifying nucleic acid” (uiNA) refers to a nucleic acid sequence comprising a guide nucleic acid (e.g., DNA or RNA) that is capable of stably associating with a nucleic acid-guided nuclease and a unique sequence identifier (e.g., barcode) that can be used to distinguish the nucleic acid from a population of nucleic acids. In some embodiments, uiNA can be operably linked to a polynucleotide (e.g., a polynucleotide encoding a test protein or a CPP-test protein fusion) or stably associated with a polypeptide to form a nucleoprotein (e.g., RNP or DNP). Accordingly, the identifier in the uiNA can be used to identify polynucleotides that have been operably linked with the uiNA, or nucleoproteins that have been stably associated with the uiNA. The sequence identifier can be located anywhere on or adjacent to the guide nucleic acid (e.g., in or adjacent to crRNA, tracrRNA, or in the tetraloop between the crRNA/trRNA on a single guide RNA). In some instances, the unique identifier is a randomized guide nucleic acid. In such embodiments, the randomized guide sequence may be one that is not capable of hybridizing with a target sequence yet can still stably associate with a nucleic acid-guided nuclease. In other embodiments, the guide nucleic acid retains its ability to hybridize with a complementary nucleic acid sequence.

The term “cell targeting agent” refers to a protein that, when associated with a nucleic acid-guided nuclease, enables at least the nucleic acid-guided nuclease (e.g., Cas9) to be targeted to the surface of a target cell or internalized by a target cell, i.e., a cell targeted by the cell targeting agent. In some embodiments, the cell targeting agent may be one that specifically binds to an extracellular target molecule (e.g., an extracellular protein or glycan) displayed on a cell membrane. In such instances, the cell targeting agent can be associated with a nucleic acid-guided nuclease such that at least the nucleic acid-guided nuclease is internalized by a target cell, i.e., a cell expressing an extracellular molecule bound by the cell targeting agent. In some embodiments, the cell targeting agent promotes internalization of the nucleic acid-guided nuclease into a membrane-bound organelle in the cell, such as the nucleus.

The terms “polypeptide” or “protein”, as used interchangeably herein, refer to any polymeric chain of amino acids. The term “polypeptide” encompasses native or artificial proteins, protein fragments and polypeptide analogs of a protein sequence.

A “test protein” refers to any protein capable of being assessed for cell targeting in accordance with the methods described herein. In some embodiments, the test protein is a protein capable of being conjugated to a nucleic acid-guided nuclease. In addition to identifying cell targeting agents that can associate with nucleic acid-guided nucleases, the methods herein are further useful for identifying variants of nucleic acid-guided nucleases (e.g., mutagenized nucleic acid-guided nucleases that have retained the ability to bind a guide nucleic acid), with or without additional agents, having desired cell targeting properties. In such cases, the nucleic acid-guided nuclease is considered the test protein.

As used herein, the term “target cell” refers to a cell or population of cells, such as mammalian cells (e.g., human cells), which includes a nucleic acid sequence in which site-directed modification of the nucleic acid is desired (e.g., to produce a genetically-modified cell). In some instances, a target cell displays on its cell membrane an extracellular molecule (e.g., an extracellular protein such as a receptor or a ligand, or glycan) specifically bound by an extracellular cell membrane binding moiety of the TAGE agent.

As used herein, the term “genetically-modified cell” refers to a cell, or an ancestor thereof, in which a DNA sequence has been deliberately modified by a site-directed modifying polypeptide (e.g., nucleic acid-guided nuclease). The term “conjugation moiety” as used herein refers to a moiety that is capable of conjugating two more or more molecules, such as a test protein and a nucleic acid-guided nuclease. The term “conjugation,” as used herein, refers to the physical or chemical complexation formed between a molecule (for e.g. a test protein) and the second molecule (e.g. a nucleic acid-guided nuclease). The chemical complexation constitutes specifically a bond or chemical moiety formed between a functional group of a first molecule (e.g., a test protein) with a functional group of a second molecule (e.g., a nucleic acid-guided nuclease). Such bonds include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. In one embodiment, conjugation is achieved via a physical association or non-covalent complexation.

As used herein, the term “ligand” refers to a molecule that is capable of specifically binding to another molecule on or in a cell, such as one or more cell surface receptors, and includes molecules such as proteins, hormones, neurotransmitters, cytokines, growth factors, cell adhesion molecules, or nutrients. A nucleic acid-guided nuclease can be associated with one or more ligands through covalent or non-covalent linkage. Examples of ligands useful herein, or targets bound by ligands, and further description of ligands in general, are disclosed in Bryant & Stow (2005). Traffic, 6(10), 947-953; Olsnes et al. (2003). Physiological reviews, 83(1), 163-182; and Planque, N. (2006). Cell Communication and Signaling, 4(1), 7, which are incorporated herein by reference.

As used herein, the term “specifically binds” refers an antigen binding polypeptide which recognizes and binds with an antigen present in a sample, but which antigen binding polypeptide does not substantially recognize or bind other molecules in the sample. In one embodiment, an antigen binding polypeptide that specifically binds to an antigen, binds to an antigen with an Kd of at least about 1×10⁻⁴, 1×10⁻⁵, 1×10⁻⁶ M, 1×10⁻⁷ M, 1×10⁻⁸ M, 1×10⁻¹⁰ M, 1×10⁻¹⁰ M, 1×10⁻¹¹ M, 1×10⁻¹² M, or more as determined by surface plasmon resonance or other approaches known in the art (e.g., filterbinding assay, fluorescence polarization, isothermal titration calorimetry), including those described further herein. In one embodiment, an antigen binding polypeptide specifically binds to an antigen if the antigen binding polypeptide binds to an antigen with an affinity that is at least two-fold greater as determined by surface plasmon resonance than its affinity for a nonspecific antigen.

The term “cell-penetrating peptide” (CPP) refers to a peptide, generally of about 5-60 amino acid residues in length, that can facilitate cellular uptake of a conjugated molecule, particularly one or more site-specific modifying polypeptides (e.g., a nucleic acid-guided nuclease). A CPP can also be characterized in certain embodiments as being able to facilitate the movement or traversal of a molecular conjugate across/through one or more of a lipid bilayer, micelle, cell membrane, organelle membrane (e.g., nuclear membrane), vesicle membrane, or cell wall. A CPP herein can be cationic, amphipathic, or hydrophobic in certain embodiments. Examples of CPPs useful herein, and further description of CPPs in general, are disclosed in Borrelli, Antonella, et al. Molecules 23.2 (2018): 295; Milletti, Francesca. Drug discovery today 17.15-16 (2012): 850-860, which are incorporated herein by reference. Further, there exists a database of experimentally validated CPPs (CPPsite, Gautam et al., 2012). The CPP can be any known CPP, such as a CPP shown in the CPPsite database.

The term “antigen binding protein” or “antigen binding polypeptide” as used herein refers to a protein that binds to a specified target antigen, such as an extracellular cell-membrane bound protein (e.g., a cell surface protein). Examples of an antigen binding polypeptide include an antibody, antigen-binding fragments of an antibody, and an antibody mimetic. In certain embodiments, an antigen-binding polypeptide is an antigen binding peptide.

The term “antibody” is used herein in the broadest sense and encompasses various antibody structures, including but not limited to monoclonal antibodies, polyclonal antibodies, multispecific antibodies (e.g., bispecific antibodies), nanobodies, monobodies, and antibody fragments so long as they exhibit the desired antigen-binding activity.

The term “antibody” includes an immunoglobulin molecule comprising four polypeptide chains, two heavy (H) chains and two light (L) chains inter-connected by disulfide bonds, as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). Each VH and VL is composed of three CDRs and four FRs, arranged from amino-terminus to carboxy-terminus in the following order: FR1, CDR1, FR2, CDR2, 1-R3, CDR3, FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

The term “Fc domain” is used to define the C-terminal region of an immunoglobulin heavy chain, which may be generated by papain digestion of an intact antibody. The Fc domain may be a native sequence Fc domain or a variant Fc domain. The Fc domain of an immunoglobulin generally comprises two constant domains, a CH2 domain and a CH3 domain, and optionally comprises a CH4 domain Replacements of amino acid residues in the Fc portion to alter antibody effector function are known in the art (Winter, et al. U.S. Pat. Nos. 5,648,260; 5,624,821). The Fc domain of an antibody mediates several important effector functions e.g. cytokine induction, ADCC, phagocytosis, complement dependent cytotoxicity (CDC) and half-life/clearance rate of antibody and antigen-antibody complexes. In certain embodiments, at least one amino acid residue is altered (e.g., deleted, inserted, or replaced) in the Fc domain of an Fc domain-containing binding protein such that effector functions of the binding protein are altered.

An “intact” or a “full length” antibody, as used herein, refers to an antibody comprising four polypeptide chains, two heavy (H) chains and two light (L) chains. In one embodiment, an intact antibody is an intact IgG antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “human antibody”, as used herein, refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of one mammalian species, such as a mouse, have been grafted onto human framework sequences. Additional framework region modifications may be made within the human framework sequences. A “humanized form” of an antibody, e.g., a non-human antibody, refers to an antibody that has undergone humanization.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

An “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv); and multispecific antibodies formed from antibody fragments.

A “multispecific antigen binding polypeptide” or “multispecific antibody” is one that targets more than one antigen or epitope. A “bispecific,” “dual-specific” or “bifunctional” antigen binding polypeptide or antibody is a hybrid antigen binding polypeptide or antibody, respectively, having two different antigen binding sites. Bispecific antigen binding polypeptides and antibodies are examples of a multispecific antigen binding polypeptide or a multispecific antibody and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553, Brinkmann and Kontermann. 2017. MABS. 9(2):182-212. The two binding sites of a bispecific antigen binding polypeptide or antibody, for example, will bind to two different epitopes, which may reside on the same or different protein targets.

The term “antibody mimetic” or “antibody mimic” refers to a molecule that is not structurally related to an antibody but is capable of specifically binding to an antigen. Examples of antibody mimetics include, but are not limited to, an adnectin (i.e., fibronectin based binding molecules), an affilin, an affimer, an affitin, an alphabody, an affibody, DARPins, an anticalin, an avimer, a fynomer, a Kunitz domain peptide, a monobody, a nanoCLAMP, a nanobody, a unibody, a versabody, an aptamer, a cyclotide, and a peptidic molecule all of which employ binding structures that, while they mimic traditional antibody binding, are generated from and function via distinct mechanisms.

Amino acid sequences described herein may include “conservative mutations,” including the substitution, deletion or addition of nucleic acids that alter, add or delete a single amino acid or a small number of amino acids in a coding sequence where the nucleic acid alterations result in the substitution of a chemically similar amino acid. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N) and glutamine (Q); N, Q, serine (S), threonine (T), and tyrosine (Y); K, R, H, D, and E; D, E, N, and Q; alanine (A), valine (V), leucine (L), isoleucine (I), proline (P), phenylalanine (F), tryptophan (W), methionine (M), cysteine (C), and glycine (G); F, W, and Y; H, F, W, and Y; C, S and T; C and A; S and T; C and S; S, T, and Y; V, I, and L; V, I, and T. Other conservative amino acid substitutions are also recognized as valid, depending on the context of the amino acid in question. For example, in some cases, methionine (M) can substitute for lysine (K). In addition, sequences that differ by conservative variations are generally homologous.

The term “isolated” refers to a compound, which can be e.g. a nucleoprotein, protein, or nucleic acid, that is substantially free of other cellular material.

As used herein, the term “operably linked” refers to polynucleotide sequences or amino acid sequences placed into a functional relationship with one another. For example, regulatory sequences (e.g., a promoter or enhancer) are “operably linked” to a polynucleotide (e.g., encoding a guide RNA or nucleic acid-guided nuclease) if the regulatory sequences regulate or contribute to the modulation of the transcription or translation of the polynucleotide. Similarly, two polypeptide-encoding nucleotide sequences are operably linked if they are contiguous and capable of expression in the same reading frame so as to produce a “fusion protein” following transcription and translation.

Additional definitions are described in the sections below.

Various aspects of the invention are described in further detail in the following subsections.

### II. Method of Identifying a Cell Targeting Agent

Provided herein are methods of identifying a cell targeting agent that, when associated with a nucleic acid-guided nuclease, enables at least the nucleic acid-guided nuclease (e.g., Cas9) to be targeted to the surface of a target cell or internalized by a target cell, i.e., a cell targeted by the cell targeting agent. In some embodiments, the cell targeting agent may be one that specifically binds to an extracellular target molecule (e.g., an extracellular protein or glycan) displayed on a cell membrane. In such instances, the cell targeting agent can be associated with a nucleic acid-guided nuclease such that at least the nucleic acid-guided nuclease is internalized by a target cell, i.e., a cell expressing an extracellular molecule bound by the cell targeting agent.

In addition to identifying cell targeting agents that can associate with nucleic acid-guided nucleases, the methods herein are further useful for identifying variants of nucleic acid-guided nucleases (e.g., mutagenized nucleic acid-guided nucleases that have retained the ability to bind a guide nucleic acid), with or without additional agents, having desired cell targeting properties. In such cases, the nucleic acid-guided nuclease is considered the test protein.

In some embodiments, the method involves providing a vector encoding (1) an RNA-guided nuclease fusion protein comprising a nucleic acid-guided nuclease (e.g., RNA-guided nuclease (e.g., RNA) or DNA-guided nuclease), or a functional fragment thereof, and a test protein, and (2) encoding a unique identifying nucleic acid (uiNA) (e.g., uiRNA or uiDNA) comprising a guide nucleic acid (e.g., gRNA or gDNA) and a sequence identifier. Examples of vectors (Section III), nucleic acid-guided nucleases (Section IV), and test proteins are described in further detail herein. In some embodiments, the method further comprises sequencing portions of the vector encoding the nucleic acid sequence identifier and the test protein are sequenced, thereby establishing an association between the test protein and identifier sequence. This association can be used to provide a reference or index for identifying the test protein based on the presence of the identifier sequence, for example, at later steps in the method.

Alternatively, the method may involve providing two or more vectors that encode the uiNA and nucleic acid-guided nuclease fusion protein, or components thereof. In instances where two vectors are used, for example, a first vector may encode a uiNA and a test agent, and a second vector may encode a nucleic acid-guided nuclease including a conjugating moiety capable of conjugating to the test agent. Upon transferring the two vectors into a same host cell, the nucleic acid-guided nuclease comprising the conjugating moiety, expressed from the second vector, and the test agent, expressed from the first vector, can stably associate to form a nucleic acid-guided nuclease fusion. The nucleic acid-guided nuclease fusion can further associate with uiNA to form a nucleoprotein.

In some embodiments, the method further involves transferring the vector to a host cell suitable to express the nucleic acid-guided nuclease fusion protein and the uiNA. In some embodiments, the vector is in a plurality of vectors and the plurality of vectors is transferred into host cells under conditions such that the average vector per host cell is 1 or more. In some embodiments, the vector is in a plurality of vectors and the plurality of vectors are transferred into host cells under conditions such that the average vector per host cell is less than 1. The nucleic acid-guided nuclease fusion protein and the uiNA can be expressed from the vector in the host cell, such that nucleoproteins (NP: e.g., DNPs or RNPs) are formed, wherein the nucleoprotein comprises the nucleic acid-guided nuclease fusion protein and the uiNA encoded on the vector. In some embodiments, the vector comprises a first promoter operatively linked to a nucleic acid sequence encoding the RNA-guided nuclease fusion protein, and comprises a second promoter operatively linked to a nucleic acid sequence encoding the uiNA. In certain embodiments, the first and second promoter are each inducible (e.g., T7 or T5) such that the expression level of the nucleic acid-guided nuclease fusion protein and the expression level of the uiNA can be controlled to obtain nucleoproteins. In some embodiments, the first and/or second promoter is a constitutive promoter.

In some embodiments, the nucleoproteins are then purified from the host cell, e.g., such that the gNA and nucleic acid-guided nuclease fusion remain stably associated following co-purification. The purified nucleoproteins can optionally be pooled together and further assessed as a pooled library of nucleoproteins, or the nucleoproteins can be assessed individually. The nucleoproteins can then be assessed for cell targeting capacity by contacting (e.g., co-incubating) the nucleoproteins with a target cell.

Accordingly, in another aspect, the method can involve providing a plurality of nucleoproteins (e.g., RNPs or DNPs) each comprising a nucleic acid-guided nuclease fusion protein and a unique identifying nucleic acid (uiNA), and proceeding with the step of contacting the nucleoproteins with a target cell, as outlined above. In some such embodiments, a reference or index may also be provided for identifying the test protein based on the presence of the identifier sequence, for example, at later steps in the method. Alternatively, the reference may be established by a variety of methods to establish the identity of the test protein and the uiNA in a nucleoprotein polypeptide.

After contacting nucleoproteins with a target cell, nucleic acids inside the target cell can be assessed to identify internalized uiNAs. In some embodiments, the method includes isolating the nucleic acids from the target cell, or a fraction thereof (e.g., cytoplasmic fraction or membrane-bound organelle fraction (e.g., nucleus, endoplasmic reticulum, Golgi apparatus, vacuole, lysosome, endosome, or mitochondria). Upon isolation, the isolated nucleic acid can be tested for the presence of the identifier sequence (e.g. by sequencing). The presence of the identifier sequence indicates that an associated test protein is a cell targeting agent. For example, identification of the test agent as a cell targeting agent may be based on a previously established reference or index establishing an association between the uiNA and the test protein in the nucleoprotein.

Following identification or selection of a test agent with the desired properties, an additional round of screening can be carried out in order to test and identify variants of the selected test agent. A sublibrary containing variants of the test agent can be created and then screened as described herein. A sublibrary refers to a library of nucleoproteins, each comprising a test agent (e.g., test cell targeting agent), that is derived from a single selected test agent or a number of test agents that is less than the number of test agents screened in the first round of selection. Variants of the test agent used for creating the sublibrary can be created or chosen by any means known in the art for creating protein variants. Production and testing of the sublibrary can be carried out by the methods outlined herein. After the sublibrary is contacted with target cells, individual variants within the sublibrary can be selected for having the desired activity. In specific embodiments, the desired activity of the identified variant can be the ability to target a nucleic acid-guided nuclease into a compartment of the target cell or binds to the cell surface of the target cell.

Additional embodiments of the methods of the invention are described in further detail herein.

Test Protein

The test protein can be any protein capable of being conjugated to a nucleic-acid guided nuclease and that can be assessed for cell targeting in accordance with the methods described herein. For example, in some embodiments, the test protein is a cell penetrating peptide (CPP). In some embodiments, the test protein is a ligand, or portion thereof. In other embodiments, the antigen-binding protein. In some embodiments, the antigen binding protein is a nanobody, a domain antibody, an scFv, a Fab, a diabody, a BiTE, a diabody, a DART, a minibody, a F(ab′)₂, an intrabody, or an antibody mimetic. In certain embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, or a versabody, an aptamer, or a cyclotide.

Test proteins can be natural, recombinant, or synthetic. In some embodiments, the test protein is one selected from a library of test proteins. In some embodiments, the test protein can be selected from a library of randomly mutated proteins. Accordingly, in some embodiments, the method can include mutagenizing a test protein (e.g., through random mutagenesis) and preparing a library of mutagenized proteins. The mutagenized test proteins can then be assessed as cell targeting agents, as described herein.

In some embodiments, a test protein is a protein or peptide found in a protein or peptide database (for example, SWISS-PROT, TrEMBL, SBASE, PFAM, CPPsite, or others known in the art), or a fragment or variant thereof. A test protein may be a protein or peptide that may be derived (for example, by transcription and/or translation) from a nucleic acid sequence known in the art, such as a nucleic acid sequence found in a nucleic acid database (for example, GenBank, TIGR, CPPsite, or others known in the art), or a fragment or variant thereof.

Unique Identifying Nucleic Acid

The unique identifying nucleic acid (uiNA) described herein includes a guide nucleic acid (e.g., DNA or RNA) that is capable of stably associating with a nucleic acid-guided nuclease and a unique sequence identifier (e.g., barcode) that can be used to distinguish the nucleic acid from a population of nucleic acids. The uiNA can be operably linked to a polynucleotide (e.g., a polynucleotide encoding a test protein or a CPP-test protein fusion) or stably associated with a polypeptide to form a nucleoprotein (e.g., RNP or DNP). Accordingly, the identifier in the uiNA can also be used to identify polynucleotides that have been operably linked with the uiNA, or nucleoproteins that have been stably associated with the uiNA.

In addition to the guide nucleic acid, the uiNA comprises a unique sequence identifier or barcode. Sequence identifiers can be any nucleic acid sequence that uniquely identifies the guide nucleic acid, and may be generated from a variety of different formats, including bulk synthesized polynucleotide barcodes, randomly synthesized barcode sequences, microarray based barcode synthesis, native nucleotides, a partial complement with an N-mer, a random N-mer, a pseudo random N-mer, or combinations thereof. In some embodiments, the sequence identifier can be a non-naturally occurring sequence. The sequence identifier can comprise, for example less than 10, 10, 11, 12, 13, 14, 15, 16, 17, 18, 19, 20, 25, 30, 35, 40, 45, 50, 55, 60, 65, 70, 75, 80, 88, 90, 100, 110, 120, 130, 140, 150, 160, 170, 180, 190, 200, or more than 200 nucleotides. Further, the sequence identifier can be located anywhere on or adjacent to the guide nucleic acid (e.g., in or adjacent to crRNA, tracrRNA, or in the tetraloop between the crRNA/trRNA on a single guide RNA). In some instances, the unique identifier is a randomized guide nucleic acid. In such embodiments, the randomized guide sequence may be one that is not capable of hybridizing with a target sequence yet can still stably associate with a nucleic acid-guided nuclease. In other embodiments, the guide nucleic acid retains its ability to hybridize with a complementary nucleic acid sequence.

The uiNA may also include additional sequence segments. Such additional sequence segments may include functional sequences, such as primer sequences, primer annealing site sequences, immobilization sequences, or other recognition or binding sequences useful for subsequent processing, e.g., a sequencing primer or primer binding site for use in sequencing of samples to which the uiNA oligonucleotide is attached.

Vector or Nucleoprotein Library

In some embodiments, the method involves producing a plurality (e.g., a library) of expression vectors, the method comprising cloning nucleic acids encoding a plurality of test proteins into an expression vector such that each expression vector contains a polynucleotide encoding a nucleic acid-guided nuclease, or a functional fragment thereof, operatively linked to at least one test protein, and a unique identifying nucleic acid (uiRNA or uiDNA), wherein the uiNA comprises a guide nucleic acid (e.g., RNA or DNA) and a sequence identifier. In some embodiments, each vector includes a single test protein. In other embodiments, each vector includes two or more test polypeptides. For example, in some embodiments, the method involves preparing a combinatorial vector library, wherein each vector encodes two or more (e.g., 2, 3, 4, 5, 6, 7, 8, 9, or 10 or more) test agents, such that nucleic acid encoding the nucleic-acid guided nuclease is operably linked to two or test agents.

In some embodiments, the library is an oligoclonal library. For example, the plasmid library can encode particular test proteins of interest and comprise replicates of plasmids encoding the same test protein. This method may be useful, for example, to optimize fractionation using a qPCR method.

In some embodiments, the method involves providing a plurality (e.g., a library) of vectors each encoding (1) a nucleic-guided nuclease fusion protein comprising a nucleic acid-guided nuclease (e.g., RNA-guided nuclease or DNA-guided nuclease), or a functional fragment thereof, and a test protein, and (2) encoding a unique identifying nucleic acid (uiNA) (e.g., uiRNA or uiDNA) comprising a guide nucleic acid (e.g., gRNA or gDNA) and a sequence identifier.

In some embodiments, the method involves producing a plurality (e.g., a library) of nucleoproteins (e.g., RNPs or DNPs), the method comprising complexing a polynucleotide encoding a nucleic acid-guided nuclease, or a functional fragment thereof, with a unique identifying nucleic acid (uiRNA or uiDNA), wherein the uiNA comprises a guide nucleic acid (e.g., RNA or DNA) and a sequence identifier. In some embodiments, each nucleoprotein includes a single test protein. In other embodiments, each nucleoprotein includes two or more test polypeptides.

In another aspect, the method may involve providing a plurality (e.g., a library) of nucleoproteins (e.g., RNPs or DNPs) each comprising a nucleic acid-guided nuclease fusion protein and a unique identifying nucleic acid (uiNA), and proceeding with the step of contacting the nucleoproteins with a target cell, as outlined above.

The plurality of vectors or nucleoproteins may be a library of vectors or nucleoproteins. The term “library” refers to a mixture of heterogeneous polypeptides or nucleic acids. The library is composed of members, which have a single polypeptide or nucleic acid sequence. Sequence differences, between library members, such as sequence differences between different test agents or uiNAs, are responsible for the diversity present in the library. The library may take the form of a simple mixture of polypeptides or nucleic acids, or may be in the form organisms or cells, for example bacteria, viruses, animal or plant cells and the like, transformed with a library of nucleic acids, such as expression vectors of the invention. Preferably, each individual organism or cell contains only one member of the library.

Vectors can be assembled from DNA encoding components of interest (e.g., a test protein, a nucleic acid-guided nuclease, a uiNA, or a regulatory element). The DNA can be obtained from any source, such as through amplification of sequences of interest from genomic DNA or through synthesis. DNA encoding a component of interest can be amplified and cloned using a known technique, such as PCR using appropriately-selected primers, in order to produce sufficient quantities of the DNA and to modify the DNA in such a manner (e.g., by addition of appropriate restriction sites) that it can be introduced as an insert into an expression vector (such as those described in Section III). Amplified and cloned DNA can be further diversified, using mutagenesis, such as PCR, in order to produce a greater diversity or wider repertoire of test proteins, as well as novel test proteins.

A cloned polynucleotide encoding any vector component described herein (e.g., a test protein, a nucleic acid-guided nuclease, a uiNA, or a regulatory element) is introduced into an expression vector (e.g., a plasmid), such as vectors described in Section III. In the case of polynucleotides encoding proteins or fusion proteins, the polynucleotide is inserted into the vector in such a manner that the protein will be expressed as protein in appropriate host cells.

In some embodiments, the method further comprises sequencing one or more portions of the vector (e.g., via plasmid-seq). For example, the method may further include sequencing one or more portions of the vector encoding the nucleic acid sequence identifier and/or the test protein, thereby establishing an association between the test protein and identifier sequence. This association can be used to provide a reference or index for identifying the test protein based on the presence of the identifier sequence, for example, at later steps in the method. For example, sequencing can be performed using automated Sanger sequencing (ABI 3730×1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; sequencing by hybridization; Sequencing with mass spectrometry; and Microfluidic Sanger sequencing. Exemplary next generating sequencing methods known to those of skill in the art include Massively parallel signature sequencing (MPSS), Polony sequencing, pyrosequencing (454), Illumina (Solexa) sequencing by synthesis, SOLiD sequencing by ligation, Ion semiconductor sequencing (Ion Torrent sequencing), DNA nanoball sequencing, chain termination sequencing (Sanger sequencing), Heliscope single molecule sequencing, Single molecule real time (SMRT) sequencing (Pacific Biosciences) and nanopore sequencing such as is described at world wide website nanoporetech.com.

These libraries of vectors are then introduced in host cells, which can be eukaryotic or prokaryotic, for expression of one or more components encoded on the vector (e.g., a test protein, a nucleic acid-guided nuclease, a nuclease-test protein fusion, and/or a uiNA). Transfer of the vector into host cells (e.g., by infection, transformation, or transfection) can be carried out using known techniques, such as electroporation, protoplast fusion, or calcium phosphate co-precipitation. In cases where the method requires two vectors, both libraries can be introduced into appropriate host cells either simultaneously or sequentially.

Compartmentalized Nucleoprotein Expression

In some embodiments, the method further involves introducing the vector into a host cell suitable to express the nucleic acid-guided nuclease fusion protein and the uiNA, and expressing the nucleic acid-guided nuclease fusion protein and the uiNA in the host cell, such that expressed nucleoproteins (NPs; RNP or DNP) each comprise a nucleic acid-guided nuclease fusion protein and the corresponding uiNA. In some embodiments, the vector is in a plurality of vectors and the plurality of vectors is transferred into host cells under conditions such that the average vector per host cell is 1 or more. In some embodiments, the vector is in a plurality of vectors and the plurality of vectors are transferred into host cells under conditions such that the average vector per host cell is less than 1. The nucleic acid-guided nuclease fusion protein and the uiNA can be expressed from the vector in the host cell, such that nucleoproteins are formed, wherein the expressed nucleoprotein comprises the nucleic acid-guided nuclease fusion protein and the uiNA encoded on the vector.

The term “host cell” refers a cell that can express proteins, protein fragments, or peptides of interest from a vector. For example, the host cell may be a prokaryotic cell or eukaryotic cell, such as a bacterial cell, an animal cell, a plant cell, or a fungal cell. In some embodiments, the eukaryotic cell is a yeast cell (e.g., a S. cerevisiae cell, Pichia pastoris, or the like), a plant cell, or mammalian cell. In some instances, the bacterial cell is an E. coli cell.

In some embodiments, the host cell is a mammalian cultured cell derived from rodents (rats, mice, guinea pigs, or hamsters) such as CHO, BHK, NSO, SP2/0, YB2/0; or human tissues or hybridoma cells, yeast cells, or insect cells. The term encompasses not only the particular subject cell but also the progeny of such a cell. Because certain modifications may occur in succeeding generations due to either mutation or environmental influences, such progeny may not be identical to the parent cell, but are still included within the scope of the term “host cell.” In certain embodiments, the mammalian cell is a COP cell, an L cell, a C127 cell, an Sp2/0 cell, an NS-0 cell, an NIH3T3 cell, a PC12 cell, a PC12h cell, a BHK cell, a CHO cell, a COS1 cell, a COS3 cell, a COST cell, a CV1 cell, a Vero cell, a HeLa cell, an HEK-293 cell, a PER C6 cell, a cell derived from diploid fibroblasts, a myeloma cell, or HepG2.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

Alternatively, the method may involve transferring the vector to a non-cellular compartment (e.g., an emulsion droplet) suitable to express the nucleic acid-guided nuclease fusion protein and the uiNA, and expressing the nucleic acid-guided nuclease fusion protein and the uiNA in the non-cellular compartment (e.g., the emulsion droplet), such that ribonucleoproteins (NPs) each comprising the nucleic acid-guided nuclease fusion protein and the uiNA are formed.

In certain embodiments, the non-cellular compartment is a droplet, such as a droplet in an emulsion and/or a microfluidic droplet. Emulsification can be used in the methods of the disclosure to separate or segregate a sample or set of samples into a series of compartments, for example a compartment having a single cell or a discrete portion of an acellular sample, such as a cell-free extract or a cell-free transcription and/or cell-free translation mixture. Typically, as used in conjunction with the methods and compositions disclosed herein, an emulsion will include a plurality of droplets, each droplet including a vector, such that each droplet includes a vector encoding one test agent and uiNA that distinguishes it from the other droplets. Emulsification can be used in the methods of the disclosure to compartmentalize one or more target molecules in emulsion droplets with one vector encoding a uiNA. Droplets in an emulsion can be sorted and/or isolated according to methods well known in the art. For example, double emulsion droplets containing a fluorescence signal can be analyzed and/or sorted using conventional fluorescence-activated cell sorting (FACS) machines at rates of >104 droplets s″1, and have been used to improve the activity of enzymes produced by single cells or by in vitro translation of single genes (Aharoni et al., Chem Biol 12(12): 1281-1289, 2005; Mastrobattista et al., Chem Biol 2(12): 1291-1300, 2005). However, the emulsions are highly polydisperse, limiting quantitative analysis, and it is difficult to add new reagents to pre-formed droplets (Griffiths et al., Trends Biotechnol 24(9):395-402, 2006). These limitations can, however, be overcome by using protocols based on droplet-based microfluidic systems (see for example Teh et al., Lab on a chip 8(2): 198-220, 2008; Theberge et al., Angew Chem Int Ed Engl 49(34):5846-5868, 2010; and Guo et al., Lab on a chip 12(12):2146, 2012) in which highly monodisperse droplets of picoliter volume can be made (Anna et al., Appl Phys Lett 82(3):364-366, 2003), fused (Song et al., Angew Chem Int Edit 42(7):767-772, 2003; Chabert et al., Electrophoresis 26(19):3706-3715, 2005), split (Song et al., Angew Chem Int Edit 42(7):767-772, 2003; Link et al., Phys Rev Lett 92(5):054503, 2004), incubated (Song et al., Angew Chem Int Edit 42(7):767-772, 2003; Frenz et al., Lab on a chip 9(10): 1344-1348, 2009), and sorted triggered on fluorescence (Baret, et al, Lab on a chip 9(13): 1850-1858, 2009), at kHz frequencies, such as those described in Mazutis et al. {Nat. Protoc. 8(5): 870-891, 2013), incorporated by reference herein. As disclosed herein, an emulsion can include various compounds, enzymes, or reagents in addition to the target molecules, target nucleic acids and origin-specific barcodes. These additives may be included in the emulsion solution prior to emulsification. Alternatively, the additives may be added to individual droplets after emulsification.

Emulsion may be achieved by a variety of methods known in the art (see, for example, US 2006/0078888 A1, of which paragraphs [0139]-[0143] are incorporated by reference herein). An exemplary emulsion is a water-in-oil emulsion. In some embodiments, the continuous phase of the emulsion includes a fluorinated oil. An emulsion can contain a surfactant or emulsifier (for example, a detergent, anionic surfactant, cationic surfactant, or amphoteric surfactant) to stabilize the emulsion. Other oil/surfactant mixtures, for example, silicone oils, may also be utilized in particular embodiments. An emulsion can be contained in a well or a plurality of wells, such as a plate, for easy of handling. In some examples, one or more vector molecules, target nucleic acid and nucleic acid barcodes are compartmentalized. An emulsion can be a monodisperse emulsion or a polydisperse emulsion. In certain embodiments, the droplet may contain an acellular system, such as a cell-free extract. The emulsion in context with the present invention may include various compounds, enzymes, or reagents in addition to the vector to achieve cell-free transcription or translation. These additives may be included in the emulsion solution prior to emulsification. Alternatively, the additives may be added to individual droplets after emulsification.

Isolation of RNPs

In some embodiments, the method further involves isolating the nucleoproteins from a host cell comprising an expression vector described herein, wherein each nucleoprotein comprises a nucleic acid-guided nuclease fusion protein and a unique identifying nucleic acid (uiNA), wherein the nucleic acid-guided nuclease fusion protein comprises a nucleic acid-guided nuclease, or a functional fragment thereof, and a test protein; and wherein the uiNA comprises a guide nucleic acid and a sequence identifier. Any purification methods can be used to isolate nucleoproteins from a host cell. Exemplary isolation techniques include, without limitation, affinity capture, immunoprecipitation, chromatography (for example, size exclusion chromatography, hydrophobic interaction chromatography, reverse-phase chromatography, ion exchange chromatography, affinity chromatography, metal binding chromatography, immunoaffinity chromatography, high performance liquid chromatography (HPLC), and liquid chromatography-mass spectrometry (LC-MS)), electrophoresis, hybridization to a capture oligonucleotide, phenol-chloroform extraction, minicolumn purification, or ethanol or isopropanol precipitation. Chromatography methods are described in detail, for example, in Hedhammar et al. (“Chromatographic methods for protein purification,” Royal Institute of Technology, Stockholm, Sweden), which is incorporated herein by reference. Such techniques can utilize a capture molecule that recognizes a labeled nucleoprotein, or a uiNA or test protein associated with the nucleoprotein.

Testing Sequence Identifiers

Isolated nucleoproteins, comprising a nucleic acid-guided nuclease fusion protein and a unique identifying nucleic acid (uiNA), can be assessed for cell targeting capacity and/or nuclear internalization capacity by contacting (e.g., co-incubating) the nucleoproteins with a target cell. For example, the contacting step may involve incubating, exposing, or mixing cells with the nucleoproteins.

In some embodiments, the target cell(s) is a eukaryotic cell, such as a mammalian cell (e.g., a human cell). In certain embodiments, the target cells are hematopoietic stem cells (HSCs), hematopoietic progenitor stem cells (HPSCs), natural killer cells, macrophages, DC cells, non-DC myeloid cells, B cells, T cells (e.g., activated T cells), fibroblasts, ocular cells, stromal cells, or other cells. In certain embodiments, the target cells are T cells. In some embodiments, the T cells are CD4 or CD8 T cells. In certain embodiments, the T cells are regulatory T cells (T regs) or effector T cells. In some embodiments, the T cells are tumor infiltrating T cells. In some embodiments, the target cell is a hematopoietic stem cell (HSC) or a hematopoietic progenitor cells (HPSCs). In some embodiments, the macrophages are M0, M1, or M2 macrophages. In some embodiments, the target cells are diseased cells. In certain embodiments, the target cells are tumor cells.

In some embodiments, Isolated nucleoproteins, comprising a nucleic acid-guided nuclease fusion protein and a uiNA, can be assessed for cell targeting capacity and/or nuclear internalization capacity by contacting (e.g., co-incubating) the nucleoproteins with multiple (e.g., 2, 3, 4, 5, 6, 7, 8, 9, 10, or more) target cells, such as multiple target cells selected from HSCs, HPSCs, natural killer cells, macrophages (e.g., M0, M1, or M2 macrophages), DC, non-DC myeloid cells, B cells, T cells (e.g., activated T cells, CD4 T cells, CD8 T cells, T regs, effector T cells, and/or tumor infiltrating T cells), fibroblasts, ocular cells, stromal cells, diseased cells (e.g., tumor cells), or other cells. In certain embodiments, isolated nucleoproteins, comprising a nucleic acid-guided nuclease fusion protein and a uiNA, can be assessed for cell targeting capacity and/or nuclear internalization capacity by contacting, such as co-incubating the nucleoproteins with multiple populations of target cells, such as a population of T cells and a population of macrophages.

The cells can be in any conditions or cell media suitable for cell viability. Further, the cells may be attached to a surface or suspended in cell media. After contacting nucleoproteins with a target cell, nucleic acids inside the target cell can then be assessed to identify internalized uiNAs.

In some embodiments, the method involves isolating the nucleic acids from the target cell, or a fraction thereof. For example, in some embodiments, the isolated nucleic acid is obtained from cytoplasm that is extracted from the target cell prior to nucleic acid isolation. Alternatively, the isolated nucleic acid is obtained from membrane-bound organelles (e.g., nucleus, endoplasmic reticulum, Golgi apparatus, vacuole, lysosome, endosome, or mitochondria) that are extracted from the target cell prior to nucleic acid isolation. For example, in some embodiments, nuclei are extracted from the target cells and the nucleic acids (e.g., including uiNA) within the extracted nuclei are isolated for further analysis. In certain embodiments, the method comprises fractionating the target cells into a first fraction comprising nuclei of the target cell and a second fraction comprising cytosol of the target cells, and the nucleic acids (e.g., including uiNA) within the extracted nuclei and extracted cytosol are isolated for further analysis.

In some embodiments, the uiNA in the original pool of nucleoproteins (the initial input prior to contacting the target cells with the nucleoproteins) is additionally assessed as a comparator. In such instances, an enrichment of the uiNA levels in the target cells, or a compartment thereof (e.g., the nucleus of the target cell) relative to the input control indicates that the associated test protein is a cell targeting agent.

In some embodiments, the method comprises contacting (e.g., via co-incubation) a mixed cell population with nucleoproteins comprising a nucleic acid-guided nuclease fusion protein and a unique identifying nucleic acid (uiNA), as described herein. In certain embodiments, the mixed cell population comprises a first cell population of cells (i.e., target cells) and a second cell population of cells (i.e., cells that are not target cells). In such instances, the method may involve isolating nucleic acids from both the first population of cells and the second population of cells. In some embodiments, the isolated nucleic acids are obtained from membrane-bound organelles in both the first population of cells and the second population of cells. Accordingly, in some embodiments, nuclei are extracted from both the first and second population of cells, and the nucleic acids (e.g., including uiNA) within the extracted nuclei are isolated for further analysis. In some embodiments, the uiNA in the original pool of nucleoproteins (the initial input prior to contacting the first and second population of cells with the nucleoproteins) is additionally assessed as a comparator. In some embodiments, the uiNA in the original pool of nucleoproteins (the initial input prior to contacting the target cells with the nucleoproteins) is additionally assessed as a comparator. In such instances, an enrichment of the uiNA levels in the target cells, or a compartment thereof (e.g., the nucleus of the target cell) relative to both the input control and the second population of cells (e.g., cells that are not target cells) indicates that the associated test protein is a cell targeting agent. In some embodiments, multiple target cell populations, such as 2, 3, 4, 5, 6, 7, 8, 9, 10, or more target cell populations described hereinabove, may be used. For example, a population of T cells and a population of macrophages can be used as target cell populations, and the uiNA in the original pool of nucleoproteins (the initial input prior to contacting the T cells and macrophages with the nucleoproteins) can be additionally assessed as a comparator. In such instances, an enrichment of the uiNA levels in the T cells and macrophages, or a compartment thereof (e.g., the nucleus of the T cells and macrophages) relative to the input control may indicate that the associated test protein is a cell targeting agent. In alternative embodiments, a population of human HSCs and a population of mouse HSCs can be used as target cell populations, and the uiNA in the original pool of nucleoproteins (the initial input prior to contacting the human HSCs and the mouse HSCs with the nucleoproteins) can be additionally assessed as a comparator. In such instances, an enrichment of the uiNA levels in the human HSCs and the mouse HSCs, or a compartment thereof (e.g., the nucleus of the human HSCs and the mouse HSCs) relative to the input control may indicate that the associated test protein is a cell targeting agent.

The nucleic acids obtained from a target cell following contact with a test nucleoprotein can be amplified for further analysis following any amplification methods known in the art. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); realtime reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others. In certain embodiments, the testing step comprises reverse-transcribing the isolated RNA to producing cDNA, and sequencing the cDNA to determine the presence of the identifier sequence. In some embodiments, the testing step comprises sequencing the isolated RNA to determine the presence of the identifier sequence.

Other exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241: 1077-1080; and Nakazawa et al. (1994) Proc. Natl. Acad. Sci. U.S.A. 91:360-364), self-sustained sequence replication (Guatelli et al. (1990) Proc. Natl. Acad. Sci. U.S.A. 87: 1874), transcriptional amplification system (Kwoh et al. (1989) Proc. Natl. Acad. Sci. U.S.A. 86:1173), Q-Beta Replicase (Lizardi et al. (1988) BioTechnology 6: 1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612, 199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

The nucleic acid (e.g., isolated nucleic acids) obtained can be tested for the presence of the identifier sequence by a variety of methods, including any sequencing or microarray methods known in the art. In some embodiments, the identity of a unique identifying nucleic acid is determined by DNA or RNA sequencing (e.g., RNA-seq). For example, the sequencing can be performed using automated Sanger sequencing (ABI 3730×1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®); Moleculo sequencing (see Voskoboynik et al. eLife 2013 2:e00569 and U.S. patent application Ser. No. 13/608,778, filed Sep. 10, 2012); DNA nanoball sequencing; Single molecule real time (SMRT) sequencing; Nanopore DNA sequencing; Sequencing by hybridization; Sequencing with mass spectrometry; and Microfluidic Sanger sequencing. Exemplary next generating sequencing methods known to those of skill in the art include Massively parallel signature sequencing (MPSS), Polony sequencing, pyrosequencing (454), Illumina (Solexa) sequencing by synthesis, SOLiD sequencing by ligation, Ion semiconductor sequencing (Ion Torrent sequencing), DNA nanoball sequencing, chain termination sequencing (Sanger sequencing), Heliscope single molecule sequencing, Single molecule real time (SMRT) sequencing (Pacific Biosciences) and nanopore sequencing such as is described at world wide website nanoporetech.com. In some embodiments, the uiNA is sequenced using a template-switch reaction (e.g., with MaximaH-Minus reverse transcriptase, derived from SMART seq, 10× Genomics), ssRNA ligation (e.g., with T4 RNA ligase K227Q, derived from microRNA seq), ssDNA ligation (e.g., with cricLigase, derived from SHAPE-seq), homopolymer tailing (e.g., with terminal transferase, derived from HTL-PCR), or splinted ligation (e.g., with T4 DNA ligase, derived from SRSLY-seq).

The presence of the identifier sequence in the target cell indicates that an associated test protein is a cell targeting agent. For example, identification of the test agent as a cell targeting agent may be based on a previously established reference or index establishing an association between the uiNA and the test protein in the nucleoprotein.

In some embodiments, the cell targeting agent identified by the present methods is a protein that targets a nucleic acid-guided nuclease into a compartment of the target cell or binds to the cell surface of the target cell. For example, the cell targeting agent compartment is a membrane-bound organelle or cytoplasm. In certain embodiments, the membrane-bound organelle is a nucleus, endoplasmic reticulum, Golgi apparatus, vacuole, lysosome, endosome, or mitochondria. In specific embodiments, internalization refers to at least 0.01%, at least 0.05%, at least 0.1%, at least 0.5%, at least 1%, at least 2%, at least 5% at least 10%, at least 15%, or at least 20% of the peptides or compositions internalized localize into the cytoplasm of a cell (e.g., within 1 hr, 2 hrs, 3 hrs, 4 hrs, or more).

### III. Expression Vectors

In another aspect, provided herein is a cell expression vector comprising: a nucleic acid encoding a nucleic acid-guided nuclease optionally operably linked to a cloning site for inserting a nucleic acid of a test protein, thereby forming a nucleic acid-guided nuclease fusion protein comprising the nucleic acid-guided nuclease and the test protein; and a nucleic acid encoding a unique identifying nucleic acid (uiNA), wherein the uiNA comprises a guide nucleic acid and a sequence identifier. In some embodiments, the expression vector further comprises the nucleic acid encoding the test protein.

“Expression vector” or “vector”, as used herein, refers to a polynucleotide vehicle that can be used to introduce genetic material into a cell. Vectors can be linear or circular. Vectors useful as expression vectors herein include plasmids, viral vectors (including phage), and integratable DNA fragments (i.e., fragments integratable into the host genome by homologous recombination). The four major types of vectors are plasmids, viral vectors, cosmids, and artificial chromosomes. Vectors can contain a replication sequence capable of effecting replication of the vector in a suitable host cell (i.e., an origin of replication). Typically, vectors comprise an origin of replication, a multicloning site, and/or a selectable marker. Upon transformation of a suitable host, the vector may replicate and function independently of the host genome or integrate into the host genome. Vector design depends, among other things, on the intended use and host cell for the vector, and the design of a vector of the invention for a particular use and host cell is within the level of skill in the art.

General methods for construction of expression vectors are known in the art. Expression vectors for most host cells are commercially available. There are several commercial software products designed to facilitate selection of appropriate vectors and construction thereof, such as bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transduction and gene expression and methods to easily enable cloning of such polynucleotides.

Expression vectors typically comprise regulatory sequences that are involved in one or more of the following: regulation of transcription, post-transcriptional regulation, and regulation of translation. Expression vectors can be introduced into a wide variety of organisms including bacterial cells, yeast cells, mammalian cells, and plant cells. Vectors typically comprise functional regulatory sequences corresponding to the host cells or organism(s) into which they are being introduced. Further, expression vectors can include polynucleotides encoding protein tags (e.g., poly-His tags, hemagglutinin tags, fluorescent protein tags, bioluminescent tags, nuclear localization tags). The coding sequences for such protein tags can be fused to the coding sequences (e.g., a sequence doing a nucleic acid-guided nuclease).

In some aspects, polynucleotides encoding one or more of the various components of the vector (e.g., a guide NA, uiNA, a nucleic acid-guided nuclease, and/or a nucleic acid-guided fusion protein) are operably linked to a promoter. For example, the operably linked promoter can be an inducible promoter, a repressible promoter, or a constitutive promoter. In some embodiments, the cell expression vector comprises a first promoter operatively linked to the nucleic acid sequence encoding the RNA-guided nuclease, and comprises a second promoter operatively linked to the nucleic acid sequence encoding the uiRNA or gRNA. In certain embodiments, the first and second promoter each comprise an inducible element such that the expression level of the RNA-guided nuclease fusion protein and the expression level of the uiRNA or gRNA can be controlled. In certain embodiments, the first and/or second promoter is T7 or T5. In some embodiments, the first and/or second promoter is a constitutive promoter.

Vectors can be designed for expression of various components of the described methods in prokaryotic or eukaryotic cells. Alternatively, transcription can be in vitro, for example using T7 promoter regulatory sequences and T7 polymerase. Other RNA polymerase and promoter sequences can be used.

Vectors can be introduced into and propagated in a prokaryote. Prokaryotic vectors are well known in the art. Typically a prokaryotic vector comprises an origin of replication suitable for the target host cell (e.g., oriC derived from E. coli, pUC derived from pBR322, pSC101 derived from Salmonella), 15A origin (derived from p15A) or bacterial artificial chromosomes). Vectors can include a selectable marker. A “selectable marker gene” refers to a gene that upon expression confers a phenotype by which successfully transformed cells carrying the vector can be identified. Selectable marker genes as used herein can confer resistance to a selection agent in cell culture and/or confer a phenotype which is identifiable upon visual inspection. In some embodiments, the selectable marker is a gene that upon expression confers resistance to a selection agent (e.g., a drug, e.g., an antibiotic, such as ampicillin, chloramphenicol, gentamicin, and kanamycin). Zeocin™ (Life Technologies, Grand Island, N.Y.) can be used as a selection in bacteria, fungi (including yeast), plants and mammalian cell lines. Accordingly, vectors can be designed that carry only one drug resistance gene for Zeocin for selection work in a number of organisms. In some embodiments, the selectable marker is a gene that upon expression confers an identifiable phenotype. For example, the selectable marker may be a fluorescent marker that confers fluorescence in cells carrying the vector that can be identified visually or by machine, e.g., flow cytometry.

Useful promoters are known for expression of proteins in prokaryotes, for example, T5, T7, Rhamnose (inducible), Arabinose (inducible), and PhoA (inducible). Further, T7 promoters are widely used in vectors that also encode the T7 RNA polymerase. Prokaryotic vectors can also include ribosome binding sites of varying strength, and secretion signals (e.g., mal, sec, tat, ompC, and pelB). In addition, vectors can comprise RNA polymerase promoters for the expression of gRNAs. Prokaryotic RNA polymerase transcription termination sequences are also well known (e.g., transcription termination sequences from S. pyogenes). Integrating vectors for stable transformation of prokaryotes are also known in the art (see, e.g., Heap, J. T., et al., “Integration of DNA into bacterial chromosomes from plasmids without a counter-selection marker,” Nucleic Acids Res. (2012) 40:e59).

Expression of proteins in prokaryotes is often carried out in bacteria, such as Escherichia coli with vectors containing constitutive or inducible promoters directing the expression of the expressed components of the vector (e.g., uiNA and nucleic acid-guided nuclease fusion protein).

A wide variety of RNA polymerase promoters suitable for expression of the various components are available in prokaryotes (see, e.g., Jiang, Y., et al., “Multigene editing in the Escherichia coli genome via the CRISPR-Cas9 system,” Environ Microbiol. (2015) 81:2506-2514); Estrem, S. T., et al., (1999) “Bacterial promoter architecture: subsite structure of UP elements and interactions with the carboxy-terminal domain of the RNA polymerase alpha subunit,” Genes Dev. 15; 13(16):2134-47).

In some aspects, a vector is a yeast expression vector comprising one or more components of the above-described methods. Examples of vectors for expression in Saccharomyces cerevisiae include, but are not limited to, the following: pYepSec1, pMFa, pJRY88, pYES2, and picZ. Methods for gene expression in yeast cells are known in the art (see, e.g., Methods in Enzymology, Volume 194, “Guide to Yeast Genetics and Molecular and Cell Biology, Part A,” (2004) Christine Guthrie and Gerald R. Fink (eds.), Elsevier Academic Press, San Diego, Calif.). Typically, expression of protein-encoding genes in yeast requires a promoter operably linked to a coding region of interest plus a transcriptional terminator. Various yeast promoters can be used to construct expression cassettes for expression of genes in yeast Examples of promoters include, but are not limited to, promoters of genes encoding the following yeast proteins: alcohol dehydrogenase 1 (ADH1) or alcohol dehydrogenase 2 (ADH2), phosphoglycerate kinase (PGK), triose phosphate isomerase (TPI), glyceraldehyde-3-phosphate dehydrogenase (GAPDH; also known as TDH3, or triose phosphate dehydrogenase), galactose-1-phosphate uridyl-transferase (GAL7), UDP-galactose epimerase (GAL10), cytochrome ci (CYC1), acid phosphatase (PHO5) and glycerol-3-phosphate dehydrogenase gene (GPD1). Hybrid promoters, such as the ADH2/GAPDH, CYC1/GAL10 and the ADH2/GAPDH promoter (which is induced at low cellular-glucose concentrations, e.g., about 0.1 percent to about 0.2 percent) also may be used. In Schizosaccharomyces pombe, suitable promoters include the thiamine-repressed nmtl promoter and the constitutive cytomegalovirus promoter in pTL2M.

Yeast RNA polymerase III promoters (e.g., promoters from 5S, U6 or RPR1 genes) as well as polymerase III termination sequences are known in the art (see, e.g., www.yeastgenome.org; Harismendy, O., et al., (2003) “Genome-wide location of yeast RNA polymerase III transcription machinery,” The EMBO Journal. 22(18):4738-4747.)

In addition to a promoter, several upstream activation sequences (UASs), also called enhancers, may be used to enhance polypeptide expression. Exemplary upstream activation sequences for expression in yeast include the UASs of genes encoding these proteins: CYC1, ADH2, GAL1, GAL7, GAL10, and ADH2. Exemplary transcription termination sequences for expression in yeast include the termination sequences of the α-factor, CYC1, GAPDH, and PGK genes. One or multiple termination sequences can be used.

Suitable promoters, terminators, and coding regions may be cloned into E. coli-yeast shuttle vectors and transformed into yeast cells. These vectors allow strain propagation in both yeast and E. coli strains. Typically, the vector contains a selectable marker and sequences enabling autonomous replication or chromosomal integration in each host. Examples of plasmids typically used in yeast are the shuttle vectors pRS423, pRS424, pRS425, and pRS426 (American Type Culture Collection, Manassas, Va.). These plasmids contain a yeast 2 micron origin of replication, an E. coli replication origin (e.g., pMB1), and a selectable marker.

The various components can also be expressed in insects or insect cells. Suitable expression control sequences for use in such cells are well known in the art. In some aspects, it is desirable that the expression control sequence comprises a constitutive promoter. Examples of suitable strong promoters include, but are not limited to, the following: the baculovirus promoters for the piO, polyhedrin (polh), p 6.9, capsid, UAS (contains a Gal4 binding site), Ac5, cathepsin-like genes, the B. mori actin gene promoter; Drosophila melanogaster hsp70, actin, α-1-tubulin or ubiquitin gene promoters, RSV or MMTV promoters, copia promoter, gypsy promoter, and the cytomegalovirus IE gene promoter. Examples of weak promoters that can be used include, but are not limited to, the following: the baculovirus promoters for the iel, ie2, ieO, etl, 39K (aka pp31), and gp64 genes. If it is desired to increase the amount of gene expression from a weak promoter, enhancer elements, such as the baculovirus enhancer element, hr5, may be used in conjunction with the promoter.

For the expression of some of the components disclosed herein in insects, RNA polymerase III promoters are known in the art, for example, the U6 promoter. Conserved features of RNA polymerase III promoters in insects are also known (see, e.g., Hernandez, G., (2007) “Insect small nuclear RNA gene promoters evolve rapidly yet retain conserved features involved in determining promoter activity and RNA polymerase specificity,” Nucleic Acids Res. 2007 January; 35(1):21-34).

In another aspect, the various components are incorporated into mammalian vectors for use in mammalian cells. A large number of mammalian vectors suitable for use with the systems of the present invention are commercially available (e.g., from Life Technologies, Grand Island, N.Y.; NeoBiolab, Cambridge, Mass.; Promega, Madison, Wis.; DNA2.0, Menlo Park, Calif.; Addgene, Cambridge, Mass.).

Vectors derived from mammalian viruses can also be used for expressing the various components of the present methods in mammalian cells. These include vectors derived from viruses such as adenovirus, papovirus, herpesvirus, polyomavirus, cytomegalovirus, lentivirus, retrovirus, vaccinia and Simian Virus 40 (SV40) (see, e.g., Kaufman, R. J., (2000) “Overview of vector design for mammalian gene expression,” Molecular Biotechnology, Volume 16, Issue 2, pp 151-160; Cooray S., et al., (2012) “Retrovirus and lentivirus vector design and methods of cell conditioning,” Methods Enzymol. 507:29-57). Regulatory sequences operably linked to the components can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are constitutive mammalian promoters CMV, EF1a, SV40, PGK1 (mouse or human), Ubc, CAG, CaMKIIa, and beta-Act. and others known in the art (Khan, K. H. (2013) “Gene Expression in Mammalian Cells and its Applications,” Advanced Pharmaceutical Bulletin 3(2), 257-263). Further, mammalian RNA polymerase III promoters, including HI and U6, can be used.

Numerous mammalian cell lines have been utilized for expression of gene products including HEK 293 (Human embryonic kidney) and CHO (Chinese hamster ovary). These cell lines can be transfected by standard methods (e.g., using calcium phosphate or polyethyleneimine (PEI), or electroporation). Other typical mammalian cell lines include, but are not limited to: HeLa, U2OS, 549, HT1080, CAD, P19, NIH 3T3, L929, N2a, Human embryonic kidney 293 cells, MCF-7, Y79, SO-Rb50, Hep G2, DUKX-X11, J558L, and Baby hamster kidney (BHK) cells. In certain embodiments, the mammalian cell is a COP cell, an L cell, a C127 cell, an Sp2/0 cell, an NS-0 cell, an NIH3T3 cell, a PC12 cell, a PC12h cell, a BHK cell, a CHO cell, a COS1 cell, a COS3 cell, a COST cell, a CV1 cell, a Vero cell, a HeLa cell, an HEK-293 cell, a PER C6 cell, a cell derived from diploid fibroblasts, a myeloma cell, or HepG2.

Methods of introducing polynucleotides (e.g., an expression vector) into host cells are known in the art and are typically selected based on the kind of host cell. Such methods include, for example, viral or bacteriophage infection, transfection, conjugation, electroporation, calcium phosphate precipitation, polyethyleneimine-mediated transfection, DEAE-dextran mediated transfection, protoplast fusion, lipofection, liposome-mediated transfection, particle gun technology, direct microinjection, and nanoparticle-mediated delivery.

### IV. Nucleic Acid-Guided Nuclease

As used herein, a “nucleic acid-guided nuclease” refers to a nuclease that is directed to a specific target sequence based on the complementarity (full or partial) between a guide nucleic acid (i.e., guide RNA or gRNA, guide DNA or gDNA, or guide DNA/RNA hybrid) that is associated with the nuclease and a target sequence. In specific embodiments, the nucleic acid-guided nuclease is a RNA guided nuclease. The binding between the guide RNA and the target sequence serves to recruit the nuclease to the vicinity of the target sequence. Non-limiting examples of nucleic acid-guided nucleases suitable for the presently disclosed compositions and methods include naturally-occurring Clustered Regularly Interspaced Short Palindromic Repeats (CRISPR)-associated (Cas) polypeptides from a prokaryotic organism (e.g., bacteria, archaea) or variants thereof. CRISPR sequences found within prokaryotic organisms are sequences that are derived from fragments of polynucleotides from invading viruses and are used to recognize similar viruses during subsequent infections and cleave viral polynucleotides via CRISPR-associated (Cas) polypeptides that function as an RNA-guided nuclease to cleave the viral polynucleotides. As used herein, a “CRISPR-associated polypeptide” or “Cas polypeptide” refers to a naturally-occurring polypeptide that is found within proximity to CRISPR sequences within a naturally-occurring CRISPR system. Certain Cas polypeptides function as RNA-guided nucleases.

There are at least two classes of naturally-occurring CRISPR systems, Class 1 and Class 2. In general, the nucleic acid-guided nucleases of the presently disclosed compositions and methods are Class 2 Cas polypeptides or variants thereof given that the Class 2 CRISPR systems comprise a single polypeptide with nucleic acid-guided nuclease activity, whereas Class 1 CRISPR systems require a complex of proteins for nuclease activity. There are at least three known types of Class 2 CRISPR systems, Type II, Type V, and Type VI, among which there are multiple subtypes (subtype II-A, II-B, II-C, V-A, V-B, V-C, VI-A, VI-B, and VI-C, among other undefined or putative subtypes). In general, Type II and Type V-B systems require a tracrRNA, in addition to crRNA, for activity. In contrast, Type V-A and Type VI only require a crRNA for activity. All known Type II and Type V RNA-guided nucleases target double-stranded DNA, whereas all known Type VI RNA-guided nucleases target single-stranded RNA. The RNA-guided nucleases of Type II CRISPR systems are referred to as Cas9 herein and in the literature. In some embodiments, the nucleic acid-guided nuclease of the presently disclosed compositions and methods is a Type II Cas9 protein or a variant thereof. Type V Cas polypeptides that function as RNA-guided nucleases do not require tracrRNA for targeting and cleavage of target sequences. The RNA-guided nuclease of Type VA CRISPR systems are referred to as Cpf1; of Type VB CRISPR systems are referred to as C2C1; of Type VC CRISPR systems are referred to as Cas12C or C2C3; of Type VIA CRISPR systems are referred to as C2C2 or Cas13A1; of Type VIB CRISPR systems are referred to as Cas13B; and of Type VIC CRISPR systems are referred to as Cas13A2 herein and in the literature. In certain embodiments, the nucleic acid-guided nuclease of the presently disclosed compositions and methods is a Type VA Cpf1 protein or a variant thereof. Naturally-occurring Cas polypeptides and variants thereof that function as nucleic acid-guided nucleases are known in the art and include, but are not limited to Streptococcus pyogenes Cas9, Staphylococcus aureus Cas9, Streptococcus thermophilus Cas9, Francisella novicida Cpf1, or those described in Shmakov et al. (2017) Nat Rev Microbiol 15(3):169-182; Makarova et al. (2015) Nat Rev Microbiol 13(11):722-736; and U.S. Pat. No. 9,790,490, each of which is incorporated herein in its entirety. Class 2 Type V CRISPR nucleases include Cas12 and any subtypes of Cas12, such as Cas12a, Cas12b, Cas12c, Cas12d, Cas12e, Cas12f, Cas12g, Cas12h, and Cas12i. Class 2 Type VI CRISPR nucleases including Cas13 can be used in order to cleave RNA target sequences.

The nucleic acid-guided nuclease of the presently disclosed compositions and methods can be a naturally-occurring nucleic acid-guided nuclease (e.g., S. pyogenes Cas9) or a variant thereof. Variant nucleic acid-guided nucleases can be engineered or naturally occurring variants that contain substitutions, deletions, or additions of amino acids that, for example, alter the activity of one or more of the nuclease domains, fuse the nucleic acid-guided nuclease to a heterologous domain that imparts a modifying property (e.g., transcriptional activation domain, epigenetic modification domain, detectable label), modify the stability of the nuclease, or modify the specificity of the nuclease.

In some embodiments, a nucleic acid-guided nuclease includes one or more mutations to improve specificity for a target site and/or stability in the intracellular microenvironment. For example, where the protein is Cas9 (e.g., SpCas9) or a modified Cas9, it may be beneficial to delete any or all residues from N175 to R307 (inclusive) of the Rec2 domain. It may be found that a smaller, or lower-molecular mass, version of the nuclease is more effective. In some embodiments, the nuclease comprises at least one substitution relative to a naturally-occurring version of the nuclease. For example, where the protein is Cas9 or a modified Cas9, it may be beneficial to mutate C80 or C574 (or homologs thereof, in modified proteins with indels). In Cas9, desirable substitutions may include any of C80A, C80L, C801, C80V, C80K, C574E, C574D, C574N, C574Q (in any combination) and in particular C80A. Substitutions may be included to reduce intracellular protein binding of the nuclease and/or increase target site specificity. Additionally, or alternatively, substitutions may be included to reduce off-target toxicity of the composition.

The nucleic acid-guided nuclease is directed to a particular target sequence through its association with a guide nucleic acid (e.g., guideRNA (gRNA), guideDNA (gDNA)). The nucleic acid-guided nuclease is bound to the guide nucleic acid via non-covalent interactions, thus forming a complex. The polynucleotide-targeting nucleic acid provides target specificity to the complex by comprising a nucleotide sequence that is complementary to a sequence of a target sequence. The nucleic acid-guided nuclease of the complex or a domain or label fused or otherwise conjugated thereto provides the site-specific activity. In other words, the nucleic acid-guided nuclease is guided to a target polynucleotide sequence (e.g. a target sequence in a chromosomal nucleic acid; a target sequence in an extrachromosomal nucleic acid, e.g. an episomal nucleic acid, a minicircle; a target sequence in a mitochondrial nucleic acid; a target sequence in a chloroplast nucleic acid; a target sequence in a plasmid) by virtue of its association with the protein-binding segment of the polynucleotide-targeting guide nucleic acid.

Thus, the guide nucleic acid comprises two segments, a “polynucleotide-targeting segment” and a “polypeptide-binding segment.” By “segment” it is meant a segment/section/region of a molecule (e.g., a contiguous stretch of nucleotides in an RNA). A segment can also refer to a region/section of a complex such that a segment may comprise regions of more than one molecule. For example, in some cases the polypeptide-binding segment (described below) of a polynucleotide-targeting nucleic acid comprises only one nucleic acid molecule and the polypeptide-binding segment therefore comprises a region of that nucleic acid molecule. In other cases, the polypeptide-binding segment (described below) of a DNA-targeting nucleic acid comprises two separate molecules that are hybridized along a region of complementarity.

The polynucleotide-targeting segment (or “polynucleotide-targeting sequence” or “guide sequence”) comprises a nucleotide sequence that is complementary (fully or partially) to a specific sequence within a target sequence (for example, the complementary strand of a target DNA sequence). The polypeptide-binding segment (or “polypeptide-binding sequence”) interacts with a nucleic acid-guided nuclease (e.g., RNA-guided nuclease). In general, site-specific cleavage or modification of the target DNA by a nucleic acid-guided nuclease occurs at locations determined by both (i) base-pairing complementarity between the polynucleotide-targeting sequence of the nucleic acid and the target DNA; and (ii) a short motif (referred to as the protospacer adjacent motif (PAM)) in the target DNA.

A protospacer adjacent motif can be of different lengths and can be a variable distance from the target sequence, although the PAM is generally within about 1 to about 10 nucleotides from the target sequence, including about 1, about 2, about 3, about 4, about 5, about 6, about 7, about 8, about 9, or about 10 nucleotides from the target sequence. The PAM can be 5′ or 3′ of the target sequence. Generally, the PAM is a consensus sequence of about 3-4 nucleotides, but in particular embodiments, can be 2, 3, 4, 5, 6, 7, 8, 9, or more nucleotides in length. Methods for identifying a preferred PAM sequence or consensus sequence for a given RNA-guided nuclease are known in the art and include, but are not limited to the PAM depletion assay described by Karvelis et al. (2015) Genome Biol 16:253, or the assay disclosed in Pattanayak et al. (2013) Nat Biotechnol 31(9):839-43, each of which is incorporated by reference in its entirety.

The unique identifying nucleic acids (uiNA) described herein comprises a guide nucleic acid sequence. The polynucleotide-targeting sequence (i.e., guide sequence) is the nucleotide sequence that directly hybridizes with the target sequence of interest. The guide sequence is engineered to be fully or partially complementary with the target sequence of interest. In various embodiments, the guide sequence can comprise from about 8 nucleotides to about 30 nucleotides, or more. For example, the guide sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the guide sequence is about 10 to about 26 nucleotides in length, or about 12 to about 30 nucleotides in length. In particular embodiments, the guide sequence is about 30 nucleotides in length. In some embodiments, the degree of complementarity between a guide sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the guide sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

In some embodiments, a guide nucleic acid comprises two separate nucleic acid molecules (an “activator-nucleic acid” and a “targeter-nucleic acid”, see below) and is referred to herein as a “double-molecule guide nucleic acid” or a “two-molecule guide nucleic acid.” In other embodiments, the subject guide nucleic acid is a single nucleic acid molecule (single polynucleotide) and is referred to herein as a “single-molecule guide nucleic acid,” a “single-guide nucleic acid,” or an “sgNA.” The term “guide nucleic acid” or “gNA” is inclusive, referring both to double-molecule guide nucleic acids and to single-molecule guide nucleic acids (i.e., sgNAs). In those embodiments wherein the guide nucleic acid is an RNA, the gRNA can be a double-molecule guide RNA or a single-guide RNA. Likewise, in those embodiments wherein the guide nucleic acid is a DNA, the gDNA can be a double-molecule guide DNA or a single-guide DNA.

An exemplary two-molecule guide nucleic acid comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the polynucleotide-targeting segment (single stranded) of the guide RNA and a stretch (“duplex-forming segment”) of nucleotides that forms one half of the dsRNA duplex of the polypeptide-binding segment of the guide RNA, also referred to herein as the CRISPR repeat sequence.

The term “activator-nucleic acid” or “activator-NA” is used herein to mean a tracrRNA-like molecule of a double-molecule guide nucleic acid. The term “targeter-nucleic acid” or “targeter-NA” is used herein to mean a crRNA-like molecule of a double-molecule guide nucleic acid. The term “duplex-forming segment” is used herein to mean the stretch of nucleotides of an activator-NA or a targeter-NA that contributes to the formation of the dsRNA duplex by hybridizing to a stretch of nucleotides of a corresponding activator-NA or targeter-NA molecule. In other words, an activator-NA comprises a duplex-forming segment that is complementary to the duplex-forming segment of the corresponding targeter-NA. As such, an activator-NA comprises a duplex-forming segment while a targeter-NA comprises both a duplex-forming segment and the DNA-targeting segment of the guide nucleic acid. Therefore, a subject double-molecule guide nucleic acid can be comprised of any corresponding activator-NA and targeter-NA pair.

The activator-NA comprises a CRISPR repeat sequence comprising a nucleotide sequence that comprises a region with sufficient complementarity to hybridize to an activator-NA (the other part of the polypeptide-binding segment of the guide nucleic acid). In various embodiments, the CRISPR repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and the antirepeat region of its corresponding tracr sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

A corresponding tracrRNA-like molecule (i.e., activator-NA) comprises a stretch of nucleotides (duplex-forming segment) that forms the other part of the double-stranded duplex of the polypeptide-binding segment of the guide nucleic acid. In other words, a stretch of nucleotides of a crRNA-like molecule (i.e., the CRISPR repeat sequence) are complementary to and hybridize with a stretch of nucleotides of a tracrRNA-like molecule (i.e., the anti-repeat sequence) to form the double-stranded duplex of the polypeptide-binding domain of the guide nucleic acid. The crRNA-like molecule additionally provides the single stranded DNA-targeting segment. Thus, a crRNA-like and a tracrRNA-like molecule (as a corresponding pair) hybridize to form a guide nucleic acid. The exact sequence of a given crRNA or tracrRNA molecule is characteristic of the CRISPR system and species in which the RNA molecules are found. A subject double-molecule guide RNA can comprise any corresponding crRNA and tracrRNA pair.

A trans-activating-like CRISPR RNA or tracrRNA-like molecule (also referred to herein as an “activator-NA”) comprises a nucleotide sequence comprising a region that has sufficient complementarity to hybridize to a CRISPR repeat sequence of a crRNA, which is referred to herein as the anti-repeat region. In some embodiments, the tracrRNA-like molecule further comprises a region with secondary structure (e.g., stem-loop) or forms secondary structure upon hybridizing with its corresponding crRNA. In particular embodiments, the region of the tracrRNA-like molecule that is fully or partially complementary to a CRISPR repeat sequence is at the 5 end of the molecule and the 3′ end of the tracrRNA-like molecule comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNC found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA-like molecule that is fully or partially complementary to the CRISPR repeat sequence comprises from about 8 nucleotides to about 30 nucleotides, or more. For example, the region of base pairing between the tracrRNA-like anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and its corresponding tracrRNA-like anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA-like molecule can comprise from about 60 nucleotides to more than about 140 nucleotides. For example, the tracrRNA-like molecule can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA-like molecule is about 80 to about 100 nucleotides in length, including about 80, about 81, about 82, about 83, about 84, about 85, about 86, about 87, about 88, about 89, about 90, about 91, about 92, about 93, about 94, about 95, about 96, about 97, about 98, about 99, and about 100 nucleotides in length.

A subject single-molecule guide nucleic acid (i.e., sgNA) comprises two stretches of nucleotides (a targeter-NA and an activator-NA) that are complementary to one another, are covalently linked by intervening nucleotides (“linkers” or “linker nucleotides”), and hybridize to form the double stranded nucleic acid duplex of the protein-binding segment, thus resulting in a stem-loop structure. The targeter-NA and the activator-NA can be covalently linked via the 3 end of the targeter-NA and the 5 end of the activator-NA. Alternatively, the targeter-NA and the activator-NA can be covalently linked via the 5′ end of the targeter-NA and the 3′ end of the activator-NA.

The linker of a single-molecule DNA-targeting nucleic acid can have a length of from about 3 nucleotides to about 100 nucleotides. For example, the linker can have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt, including but not limited to about 3, about 4, about 5, about 6, about 7, about 8, about 9, about 10, about 11, about 12, about 13, about 14, about 15, about 16, about 17, about 18, about 19, about 20, or more nucleotides. In some embodiments, the linker of a single-molecule DNA-targeting nucleic acid is 4 nt.

An exemplary single-molecule DNA-targeting nucleic acid comprises two complementary stretches of nucleotides that hybridize to form a double-stranded duplex, along with a guide sequence that hybridizes to a specific target sequence.

Appropriate naturally-occurring cognate pairs of crRNAs (and, in some embodiments, tracrRNAs) are known for most Cas proteins that function as nucleic acid-guided nucleases that have been discovered or can be determined for a specific naturally-occurring Cas protein that has nucleic acid-guided nuclease activity by sequencing and analyzing flanking sequences of the Cas nucleic acid-guided nuclease protein to identify tracrRNA-coding sequence, and thus, the tracrRNA sequence, by searching for known antirepeat-coding sequences or a variant thereof. Antirepeat regions of the tracrRNA comprise one-half of the ds protein-binding duplex. The complementary repeat sequence that comprises one-half of the ds protein-binding duplex is called the CRISPR repeat. CRISPR repeat and antirepeat sequences utilized by known CRISPR nucleic acid-guided nucleases are known in the art and can be found, for example, at the CRISPR database on the world wide web at crispr.i2bc.paris-saclay.fr/crispr/.

The single guide nucleic acid or dual-guide nucleic acid can be synthesized chemically or via in vitro transcription. Assays for determining sequence-specific binding between a nucleic acid-guided nuclease and a guide nucleic acid are known in the art and include, but are not limited to, in vitro binding assays between an expressed nucleic acid-guided nuclease and the guide nucleic acid, which can be tagged with a detectable label (e.g., biotin) and used in a pull-down detection assay in which the nucleoprotein complex is captured via the detectable label (e.g., with streptavidin beads). A control guide nucleic acid with an unrelated sequence or structure to the guide nucleic acid can be used as a negative control for non-specific binding of the nucleic acid-guided nuclease to nucleic acids.

In addition to the guide nucleic acid, the uiNA comprises a unique sequence identifier or barcode. Sequence identifiers can be any nucleic acid sequence that uniquely identifies the guide nucleic acid, and may be generated from a variety of different formats, including bulk synthesized polynucleotide barcodes, randomly synthesized barcode sequences, microarray based barcode synthesis, native nucleotides, a partial complement with an N-mer, a random N-mer, a pseudo random N-mer, or combinations thereof. In some embodiments, the sequence identifier can be a non-naturally occurring sequence. The sequence identifier can comprise, for example less than 10, 10, 11, 12, 13, 14, 15, 16, 17, 18, 19, 20, 25, 30, 35, 40, 45, 50, 55, 60, 65, 70, 75, 80, 88, 90, 100, 110, 120, 130, 140, 150, 160, 170, 180, 190, 200, or more than 200 nucleotides. Further, the sequence identifier can be located anywhere on or adjacent to the guide nucleic acid (e.g., in or adjacent to crRNA, tracrRNA, or in the tetraloop between the crRNA/trRNA on a single guide RNA). In some instances, the unique identifier is a randomized guide nucleic acid. In such embodiments, the randomized guide sequence may be one that is not capable of hybridizing with a target sequence yet can still stably associated with a nucleic acid-guided nuclease. In other embodiments, the guide nucleic acid retains its ability to hybridize with a complementary nucleic acid sequence.

The uiNA may also include additional sequence segments. Such additional sequence segments may include functional sequences, such as primer sequences, primer annealing site sequences, immobilization sequences, or other recognition or binding sequences useful for subsequent processing, e.g., a sequencing primer or primer binding site for use in sequencing of samples to which the uiNA oligonucleotide is attached.

In certain embodiments, the nucleic acid-guided nuclease of the presently disclosed compositions and methods comprise a nuclease variant that functions as a nickase, wherein the nuclease comprises a mutation in comparison to the wild-type nuclease that results in the nuclease only being capable of cleaving a single strand of a double-stranded nucleic acid molecule, or lacks nuclease activity altogether (i.e., nuclease-dead).

A nuclease, such as a nucleic acid-guided nuclease, that functions as a nickase only comprises a single functioning nuclease domain. In some of these embodiments, additional nuclease domains have been mutated such that the nuclease activity of that particular domain is reduced or eliminated.

In other embodiments, the nuclease (e.g., RNA-guided nuclease) lacks nuclease activity completely and is referred to herein as nuclease-dead. In some of these embodiments, all nuclease domains within the nuclease have been mutated such that all nuclease activity of the polypeptide has been eliminated. Any method known in the art can be used to introduce mutations into one or more nuclease domains of a nucleic acid-guided nuclease, including those set forth in U.S. Publ. Nos. 2014/0068797 and U.S. Pat. No. 9,790,490, each of which is incorporated by reference in its entirety.

Any mutation within a nuclease domain that reduces or eliminates the nuclease activity can be used to generate a nucleic acid-guided nuclease having nickase activity or a nuclease-dead nucleic acid-guided nuclease. Such mutations are known in the art and include, but are not limited to the D10A mutation within the RuvC domain or H840A mutation within the HNH domain of the S. pyogenes Cas9 or at similar position(s) within another nucleic acid-guided nuclease when aligned for maximal homology with the S. pyogenes Cas9. Other positions within the nuclease domains of S. pyogenes Cas9 that can be mutated to generate a nickase or nuclease-dead protein include G12, G17, E762, N854, N863, H982, H983, and D986. Other mutations within a nuclease domain of a nucleic acid-guided nuclease that can lead to nickase or nuclease-dead proteins include a D917A, E1006A, E1028A, D1227A, D1255A, N1257A, D917A, E1006A, E1028A, D1227A, D1255A, and N1257A of the Francisella novicida Cpf1 protein or at similar position(s) within another nucleic acid-guided nuclease when aligned for maximal homology with the F. novicida Cpf1 protein (U.S. Pat. No. 9,790,490, which is incorporated by reference in its entirety).

Nucleic acid-guided nucleases comprising a nuclease-dead domain can further comprise a domain capable of modifying a polynucleotide. Non-limiting examples of modifying domains that may be fused to a nuclease-dead domain include but are not limited to, a transcriptional activation or repression domain, a base editing domain, and an epigenetic modification domain. In other embodiments, the nucleic acid-guided nuclease comprising a nuclease-dead domain further comprises a detectable label that can aid in detecting the presence of the target sequence.

An epigenetic modification domain that can be fused to a nuclease-dead domain can serve to covalently modify DNA or histone proteins to alter histone structure and/or chromosomal structure without altering the DNA sequence itself, leading to changes in gene expression (upregulation or downregulation). Non-limiting examples of epigenetic modifications that can be induced by nucleic acid-guided nuclease include the following alterations in histone residues and the reverse reactions thereof: sumoylation, methylation of arginine or lysine residues, acetylation or ubiquitination of lysine residues, phosphorylation of serine and/or threonine residues; and the following alterations of DNA and the reverse reactions thereof: methylation or hydroxymethylation of cytosine residues. Non-limiting examples of epigenetic modification domains thus include histone acetyltransferase domains, histone deacetylation domains, histone methyltransferase domains, histone demethylase domains, DNA methyltransferase domains, and DNA demethylase domains.

In some embodiments, the nucleic acid-guided nuclease comprises a transcriptional activation domain that activates the transcription of at least one adjacent gene through the interaction with transcriptional control elements and/or transcriptional regulatory proteins, such as transcription factors or RNA polymerases. Suitable transcriptional activation domains are known in the art and include, but are not limited to, VP16 activation domains.

In other embodiments, the nucleic acid-guided nuclease comprises a transcriptional repressor domain, which can also interact with transcriptional control elements and/or transcriptional regulatory proteins, such as transcription factors or RNA polymerases, to reduce or terminate transcription of at least one adjacent gene. Suitable transcriptional repression domains are known in the art and include, but are not limited to, IκB and KRAB domains.

In still other embodiments, the nucleic acid-guided nuclease comprising a nuclease-dead domain further comprises a detectable label that can aid in detecting the presence of the target sequence, which may be a disease-associated sequence. A detectable label is a molecule that can be visualized or otherwise observed. The detectable label may be fused to the nucleic-acid guided nuclease as a fusion protein (e.g., fluorescent protein) or may be a small molecule conjugated to the nuclease polypeptide that can be detected visually or by other means. Detectable labels that can be fused to the presently disclosed nucleic-acid guided nucleases as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

The nucleic acid-guided nuclease can be delivered as part of a fusion protein (e.g., RNA-guided nuclease fusion protein) into a cell as a nucleoprotein complex comprising the nucleic acid-guided nuclease bound to its guide nucleic acid. Alternatively, the nucleic acid-guided nuclease is delivered as a fusion protein and the guide nucleic acid is provided separately. In certain embodiments, a guide RNA can be introduced into a target cell as an RNA molecule. The guide RNA can be transcribed in vitro or chemically synthesized. In other embodiments, a nucleotide sequence encoding the guide RNA is introduced into the cell. In some of these embodiments, the nucleotide sequence encoding the guide RNA is operably linked to a promoter (e.g., an RNA polymerase III promoter), which can be a native promoter or heterologous to the guide RNA-encoding nucleotide sequence. In specific embodiments, a nucleic acid sequence encoding the guide RNA and RNA-guided nuclease operably linked to a promoter can be delivered on a vector, such as the expression vector described in detail herein.

In certain embodiments, the nucleic acid-guided nuclease fusion protein can comprise additional amino acid sequences, such as at least one nuclear localization sequence (NLS). Nuclear localization sequences enhance transport of the nucleic acid-guided nuclease into the nucleus of a cell. Proteins that are imported into the nucleus bind to one or more of the proteins within the nuclear pore complex, such as importin/karypherin proteins, which generally bind best to lysine and arginine residues. The best characterized pathway for nuclear localization involves short peptide sequence which binds to the importin-α protein. These nuclear localization sequences often comprise stretches of basic amino acids and given that there are two such binding sites on importin-α, two basic sequences separated by at least 10 amino acids can make up a bipartite NLS. The second most characterized pathway of nuclear import involves proteins that bind to the importin-β1 protein, such as the HIV-TAT and HIV-REV proteins, which use the sequences RKKRRQRRR (SEQ ID NO: 23) and RQARRNRRRRWR (SEQ ID NO: 24), respectively to bind to importin-β1. Other nuclear localization sequences are known in the art (see, e.g., Lange et al., J. Biol. Chem. (2007) 282:5101-5105). The NLS can be the naturally-occurring NLS of the nucleic acid-guided nuclease or a heterologous NLS. As used herein, “heterologous” in reference to a sequence is a sequence that originates from a foreign species, or, if from the same species, is substantially modified from its native form in composition and/or genomic locus by deliberate human intervention. Non-limiting examples of NLS sequences that can be used to enhance the nuclear localization of the nucleic acid-guided nuclease or nucleic acid-guided nuclease fusion protein include the NLS of the SV40 Large T-antigen and c-Myc. In certain embodiments, the NLS comprises the amino acid sequence PKKKRKV (SEQ ID NO: 25).

A nucleic acid-guided nuclease fusion protein can comprise more than one NLS, such as two, three, four, five, six, or more NLS sequences. Each of the multiple NLSs can be unique in sequence or there can be more than one of the same NLS sequence used. The NLS can be on the amino-terminal (N-terminal) end of the nucleic acid-guided nuclease fusion protein, the carboxy-terminal (C-terminal) end, or both the N-terminal and C-terminal ends of the fusion protein. In certain embodiments, the nucleic acid-guided nuclease fusion protein comprises two NLS sequences on its N-terminal end. In other embodiments, the nucleic acid-guided nuclease fusion protein comprises two NLS sequences on the C-terminal end of the site-directed polypeptide. In still other embodiments, the site-directed polypeptide comprises four NLS sequences on its N-terminal end and two NLS sequences on its C-terminal end.

In some embodiments, the nucleic acid-guided nuclease fusion protein can comprise an epitope tag. For example, an epitope tag may be a poly-histidine tag such as a hexahistidine tag (SEQ ID NO: 22) or a dodecahistidine (SEQ ID NO: 26), a FLAG tag, a Myc tag, a HA tag, a GST tag or a V5 tag. In particular embodiments, the nucleic acid-guided nuclease fusion protein comprises from 5 to 3 hexahistidine tag (6×His) (SEQ ID NO: 22), a test protein (e.g., CPP, or variant thereof), Cas9, and 2×NLS.

In certain embodiments, the nucleic acid-guided nuclease fusion protein comprises a test protein, or variant thereof. The test protein can be any protein, or variant thereof, to be tested using the methods and compositions described herein.

In some embodiments, the test protein is a cell penetrating peptide (CPP), which induces the absorption of a linked protein or peptide through the plasma membrane of a cell. Generally, CPPs induce entry into the cell because of their general shape and tendency to either self-assemble into a membrane-spanning pore, or to have several positively charged residues, which interact with the negatively charged phospholipid outer membrane inducing curvature of the membrane, which in turn activates internalization. Exemplary permeable peptides include, but are not limited to, transportan, PEP1, MPG, p-VEC, MAP, CADY, polyR, HIV-TAT, HIV-REV, Penetratin, R6W3, P22N, DPV3, DPV6, K-FGF, and C105Y, and are reviewed in van den Berg and Dowdy (2011) Current Opinion in Biotechnology 22:888-893 and Farkhani et al. (2014) Pepides 57:78-94, each of which is herein incorporated by reference in its entirety.

Along with or as an alternative to an NLS, the nucleic acid-guided nuclease fusion protein can comprise additional heterologous amino acid sequences, such as a detectable label (e.g., fluorescent protein) described elsewhere herein, or a purification tag, to form a fusion protein. A purification tag is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

### EXAMPLES

The invention will be more fully understood by reference to the following examples. They should not, however, be construed as limiting the scope of the invention. All literature and patent citations are incorporated herein by reference.

### Example 1—High Throughput Cloning of CPP-Cas9 Library

Examples 1-4 relate to a screen designed to rapidly assay a pool of Cas9-fusion proteins including different test cell penetrating peptides (CPP) for CPPs that effectively facilitate internalization of a Cas9. A plurality of unique identifying RNAs (uiRNA) including a guide RNA (gRNA) and a library of polynucleotides encoding over 3000 different test CPPs were combinatorially assembled into a vector library encoding Cas9. The vectors were assembled such that the CPP was operably linked to Cas9. By sequencing the vector, the uiRNA and test CPP on each vector were identified, thereby providing a reference of pairs of associated uiRNA and test CPPs that could be used to identify CPP-Cas9 ribonucleoproteins based on the presence of the uiRNA at later steps.

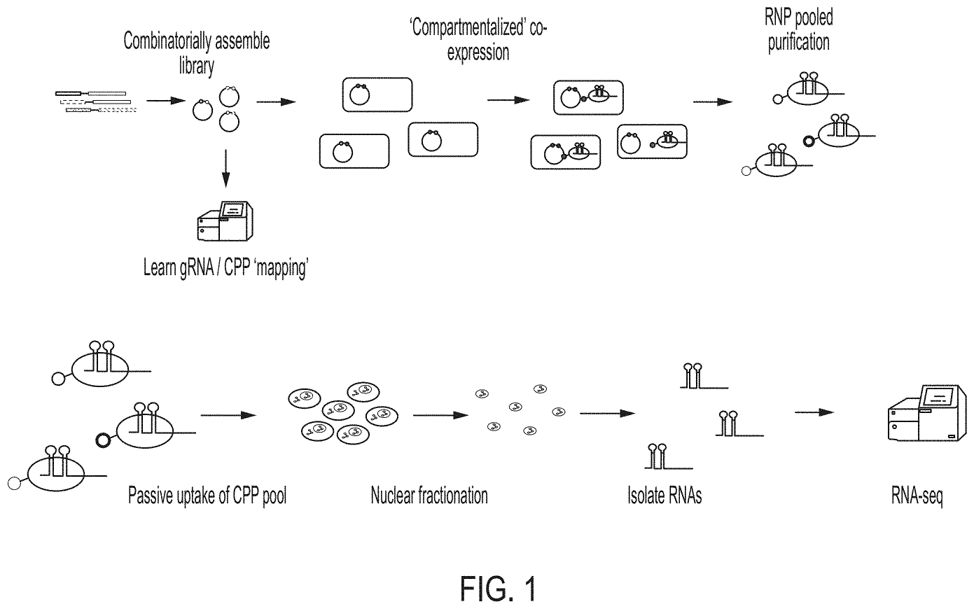

The plasmid library was transformed into E. coli, in which compartmentalized expression of the CPP-Cas9 fusion and uiRNA enabled formation of CPP-Cas9 RNPs (i.e., comprising the uiRNA and the CPP-Cas9 fusion previously established as being paired on a single library vector). The CPP-Cas9 fusions were isolated from the bacterial cells to generate a pool of CPP-Cas9 RNPs. The pooled CPP-Cas9 RNPs were then assessed for cellular targeting by co-incubation with target cells. Following co-incubation, nuclear fractionation was performed on the target cells and RNA was isolated and sequenced from the nuclear extractions. The uiRNAs identified in the isolated nuclear RNAs were used to identify candidate CPPs that effectively facilitated cellular uptake of Cas9. A flowchart summarizing the general workflow of this screen is shown in FIG. 1.

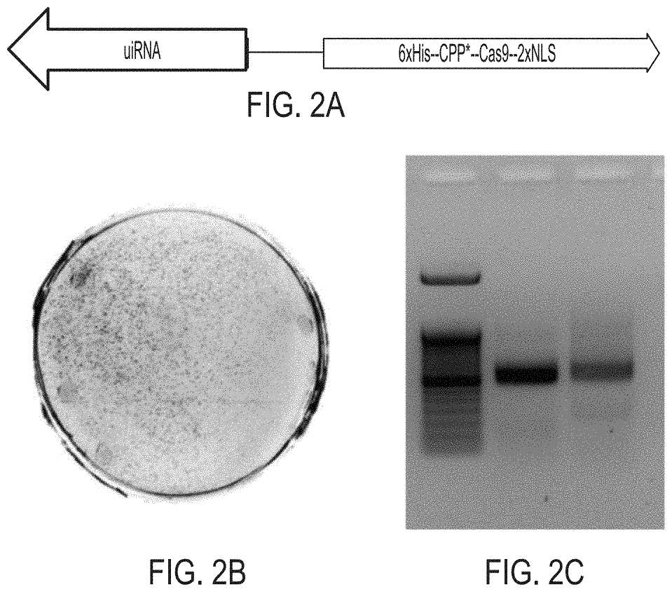

First, to combinatorially assemble the vector library, a modular plasmid was constructed containing a uiRNA cassette and a Cas9 homolog operably linked to a test CPP randomly selected from a library of approximately 3200 unique test CPPs computationally identified from existing databases for NLS and CPP peptides. The specific test protein can be readily swapped with any test protein of interest. Likewise, the Cas9 homolog can be readily exchanged with any other nucleic acid-guided nuclease of interest. The sgRNA cassette of the constructs included a T7 promoter, sgRNA (with or without a random barcode), 3′ HDV ribozyme, and a 3′ RRNB terminator. An exemplary map of a nucleic acid encoding a uiRNA linked to a CPP-encoding nucleotide is shown in FIG. 2A. The plasmid further encoded a His6 tag (SEQ ID NO: 22) to aid in purification of a CPP-Cas9 fusion, a HRV 3c protease site, and the modular site for insertion of the CPP at the N-terminus of the polynucleotide encoding Cas9 (C80A).

Each component of the plasmid was prepared by PCR amplification prior to plasmid assembly. The vector backbone and Cas9 (C80A) was PCR amplified using Golden Gate cloning primers. To design a pool of oligonucleotides encoding different CPPs, each test CPP from the library of CPPs was reverse translated and codon optimized in silico, DNA hairpins were removed, primer binding sites were added, and synthetic oligos were ordered for each CPP. The CPP-encoding oligonucleotide pool was then PCR amplified and inserted into expression vectors. A uiRNA block (including promoter, variable sgRNA portion, HDV, and a RRNB terminator) prepared from gBlocks or ultramer synthetic DNAs was PCR amplified. All PCR amplification was performed with Q5 2× Master Mix (New England Bio) at a volume of 50 ul and was carried out for 35 cycles. All PCR reactions had a primer concentration of 1 μM with an annealing temperature of 60° C., and the primers were annealed for 15 s. Extension was performed at 72° C. for 1 minute for all constructs, except for the Cas9 PCR amplification (3 minutes at 72° C.) and the vector backbone (5 minutes at 72° C.). After PCR amplification, all products were purified by Zymo DNA Clean and Concentrate kit and verified by visualizing the products on an agarose gel by gel electrophoresis.

Next, the CPP-encoding oligonucleotide pool, uiRNA blocks, and promoter cassettes were assembled by overlap extension PCR. 250 ng of each insert was mixed in a 50 μl Q5 master mix reaction. The reaction was then thermocycled, without the addition of primers, following standard temperatures and times (60° C. annealing for 15 s, 72° C. for 1 minute) and then purified using the Zymo DNA Clean and Concentrate kit.

A polynucleotide cassette encoding Cas9 (C80A) was then amplified using PCR primers that enable Golden Gate cloning. Next, the expression vector was assembled by mixing 2.5 μg of the Cas9 (C80) PCR product with 2.5 μg of the vector PCR product, and 500 ng of the overlap extension insert, and assembled with standard Golden Gate cloning using Sap I type IIS restriction enzyme and T4 DNA ligase. The assembled constructed was electro-transformed into a cloning E. coli strain (Top10 or NebTurbo) following the manufacture's protocol. An exemplary agar plate containing colonies from a library of approximately 5000 E. coli transformants is shown in FIG. 2B. The plasmid library was harvested from the transformants using a Qiagen Midi-prep kit. The results of a gel electrophoresis analysis of the isolated plasmid library (two replicates) is provided in FIG. 2C. The plasmid library was further assessed as outlined in Example 2.

### Example 2—gRNA/CPP Mapping of CPP-Cas9 Library

The plasmid library encoding uiRNA and CPP-Cas9 fusions, prepared as outlined in Example 1, was then prepared for next generation sequencing (NSG) on an Illumina sequencer following the workflow below.

A unique molecular identifier (UMI), which controls for PCR bias, was added by 2-cycle PCR amplification of the plasmid library. UMI primers (1 μM) were mixed with the primer pool (10 ng plasmid; ˜10⁻⁹ molecules) in 50 μl Q5 master mix. The mixture was then thermocycled following a standard protocol for 2 cycles. 1 μL Exol was added to the reaction to degrade excess primers. Exol was then heat inactivated by incubating the sample at 80° C.

Exponential PCR amplification was performed to add Illumina sequencing adaptors, using the standard manufacturer's protocol (annealing temperature of 65° C.). The PCR products were gel-purified, and sequenced on an Illumina MiSeq sequencer with a 150 cycle kit.

The pooled plasmid data was analyzed by custom scripts. The read was split into various fields based on UMIs, barcoded uiRNA, and the Cas9 CPP fusion. UMIs were counted to account for PCR bias, and reads with duplicate UMIs were discarded.

The CPP-Cas9 fusion was then assigned to a particular barcoded uiRNA by aligning the CPP-Cas9 fusion to the CPP-encoding oligonucleotide using Bowtie2 aligner. A map associating the CPP-Cas9 fusion to each uiRNA barcode was built by parsing the alignment and the uiRNA field. A table was prepared that maps each uiRNA to a particular CPP-Cas9 fusion identified on each vector.

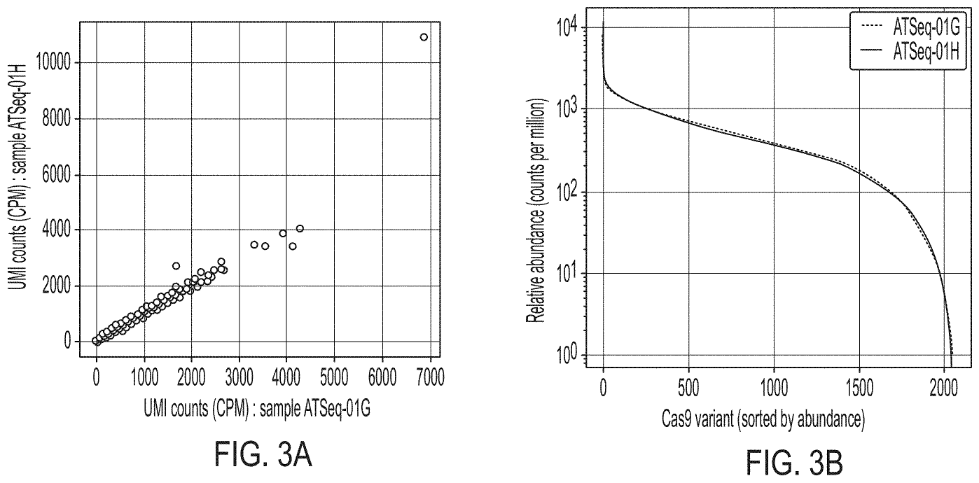

The data was reproducible, as shown in FIG. 3A, which compares the plasmid-seq UMI counts between replicates. 2000 test CPPs were observed in the vector library out of the original 3400 (˜58% coverage) in the original pool of CPP-encoding oligonucleotides. FIG. 3B graphically depicts the library coverage distribution for the CPP-Cas9 fusions from each run, showing that the relative abundance of different CPP-fusions was biased. To identify potential sources of plasmid non-uniformity, the number of UMI counts per Cas9-CPP fusion, number of guides per Cas9-CPP fusion, and number of UMIs per uiRNA was assessed.

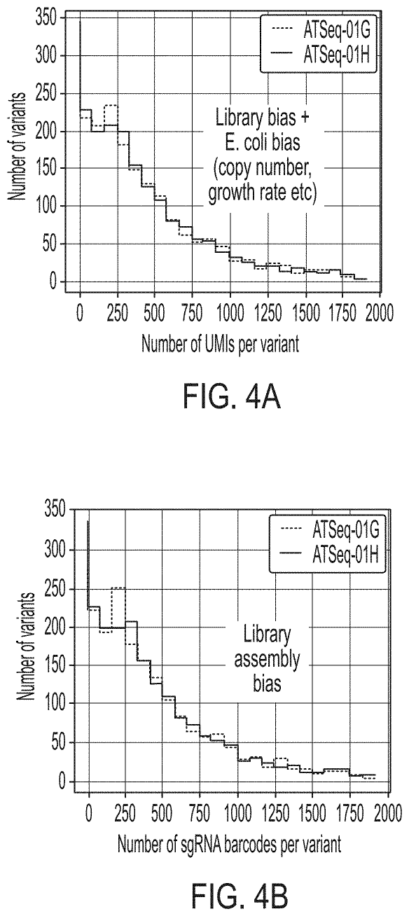

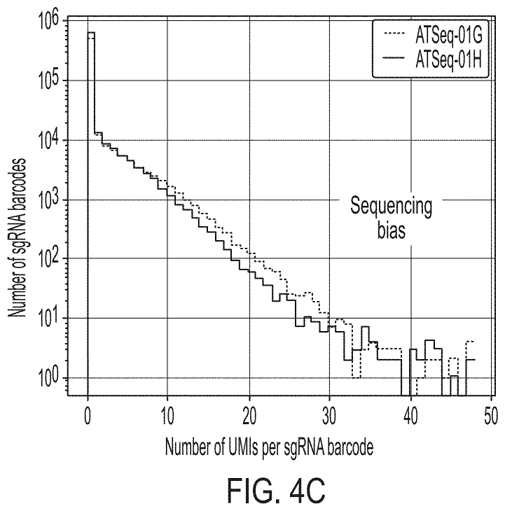

FIG. 4A graphically depicts the number of plasmid UMIs per CPP-Cas9 fusion for two library replicates, which is indicative of library bias or cloning bias in E. coli (e.g., due to differences in copy number or growth rate). Most variants have few UMIs per variant, but a small number of variants have a large number of UMIs. This indicates that there are a small number of variants (i.e., different CPP-Cas9 fusions) that are overrepresented in the plasmid pool.

FIG. 4B graphically depicts the number of sgRNA barcodes (i.e., uiRNA) per CPP-Cas9 fusion, which is indicative of library assembly bias (e.g., during PCR or overlap assembly steps). Most variants (i.e., different CPP-Cas9 fusions) have a few distinct sgRNA barcodes, but a few variants have several distinct sgRNA barcodes associated with them. This implies that the root cause of bias shown in FIG. 4A has occurred before the randomized sgRNA barcode was ligated to the Cas9 vector. The most likely conclusion for this is that the underlying oligo pool which encodes the different CPP-Cas9 fusions was skewed to begin with.

FIG. 4C graphically depicts the number of UMIs per sgRNA barcodes (i.e., uiRNA), which is indicative of sequencing bias. The sequencing library was prepared with unique molecular identifiers (UMIs) which were used to account for PCR bias. These results show that PCR bias is not significant (note log scale y axis), and has been accounted for. This makes conclusions of FIG. 4A and FIG. 4B more quantitative.

In summary, these results show that the modular high-throughput cloning strategy works, and enables preparation of plasmid libraries encoding a library of different Cas9-CPP fusions that can be purified and assessed, as further outlined in Example 3 and Example 4.

### Example 3—Purification of Candidate CPP-Cas9 RNPs from Library

Next, the plasmid library was transformed into E. coli, in which compartmentalized expression of the CPP-Cas9 fusion and uiRNA enabled formation of CPP-Cas9 RNPs (i.e., comprising the uiRNA and the CPP-Cas9 fusion previously established as being paired on a single library vector). Expression of the CPP-Cas9 fusion was driven by the T5/lac inducible promoter and uiRNA expression was driven by the T7 promoter. In BL21DE cells, the expression of T7 RNA polymerase was also lac inducible. Therefore, the addition of IPTG will result in expression of both Cas9 and uiRNAs simultaneously.

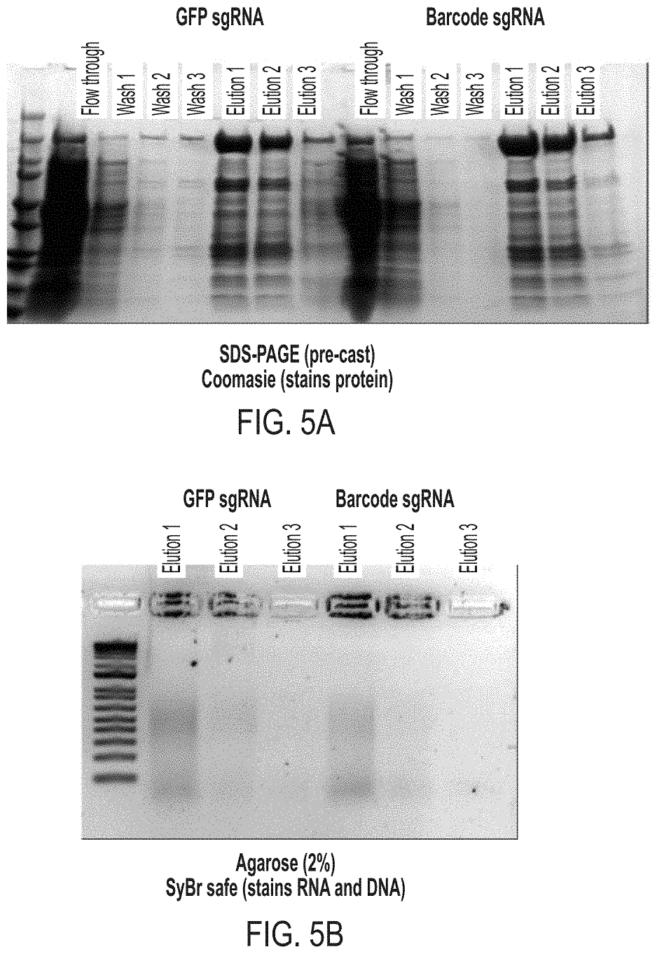

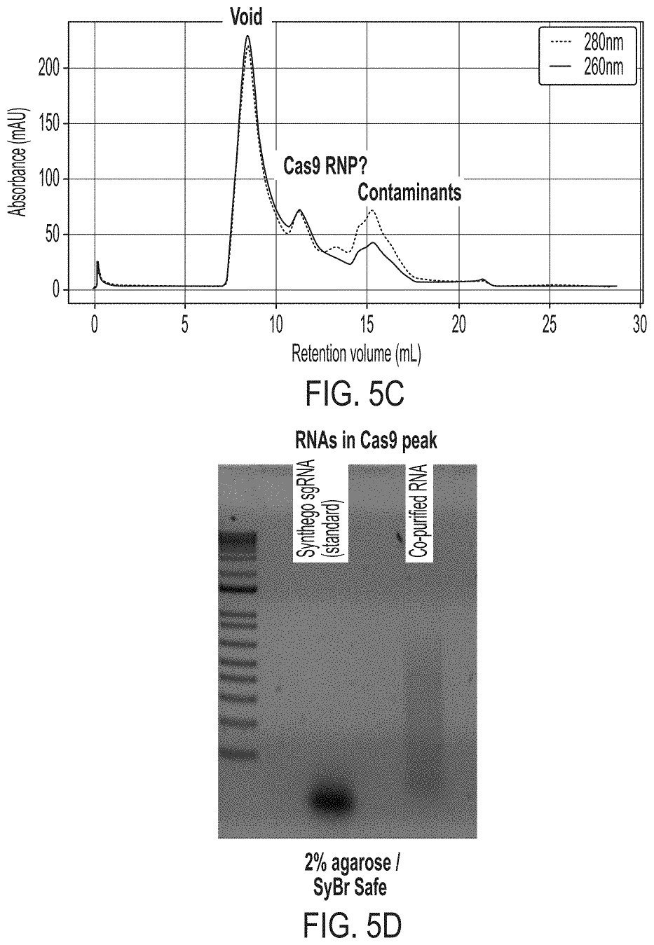

1 L of E. coli transformed with the plasmid library was grown for 2-5 hours at 37° C. until reaching an optical density of OD1. CPP-Cas9 fusion expression and uiRNA expression was induced by adding 1 mM IPTG. The temperature was then dropped to 16° C. and the culture was grown overnight for 16-20 hours. E. coli cells were pelleted and lysed by sonication. His6×-CPP-Cas9 (“His6×” disclosed as SEQ ID NO: 22) RNPs were affinity purified using a nickel resin, and eluted form the resin with imidazole. The affinity-purified nucleoproteins were validated by SDS-PAGE analysis (FIG. 5A) and gel electrophoresis (FIG. 5B). CPP-Cas9 RNPs were further purified by size exclusion chromatography using an ACTA FPLC and an S200 column (FIG. 5C). Bulk RNAs were phenol extracted from the RNPs and analyzed by gel electrophoresis (2% agarose, SyBr Safe dye), as shown in FIG. 5D, confirming the presence of co-purified RNAs extracted from the purified RNPs.

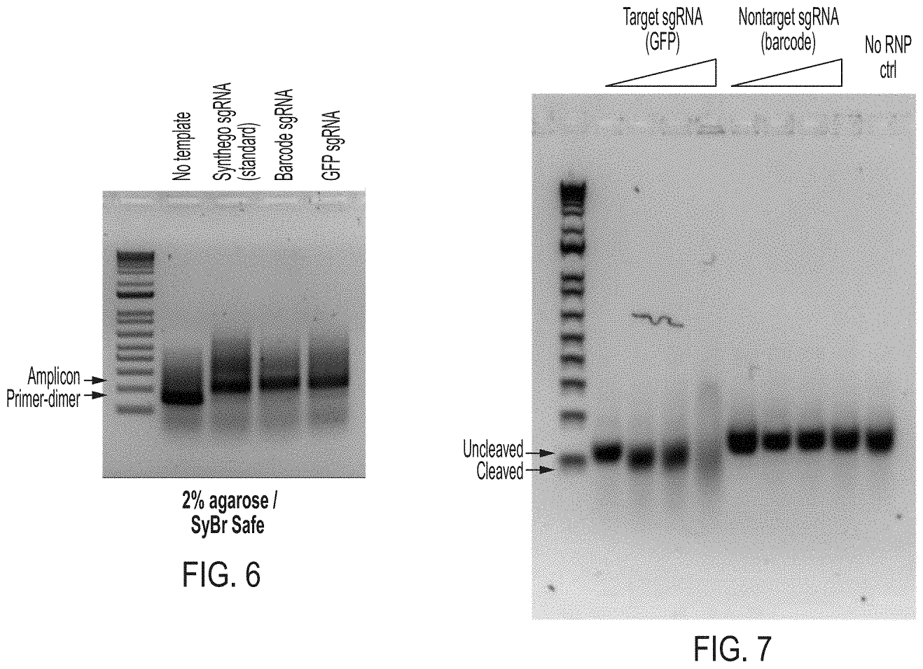

To verify the identity of RNAs that were co-purified with the RNPs, co-purified RNA was amplified by template-switch reverse transcription. A guide-specific reverse transcription primer was used with a template switch at the 5′end of the template. This adds a second primer binding sequence and adds an UMI. FIG. 6 shows an image of a gel electrophoresis analysis (2% agarose gel, SyBr Safe dye) of RNA samples amplified by reverse-transcription. The results indicate that uiRNA or GFP sgRNA was successfully co-purified with the RNPs.

Next, purified CPP-Cas9 fusions from the library were assessed for catalytic activity. 0 uM, 1 uM, 2 uM, or 10 uM of Cas9 RNP having target sgRNA (GFP) and nontarget sgRNA (uiRNA) were incubated with dsDNA at 37° C. for 30 minutes. FIG. 7 shows an image of a gel electrophoresis analysis of samples from the DNA cleavage assay. Bands corresponding to uncleaved and cleaved dsRNA are indicated. dsRNA from a no RNP control condition is also shown. Target DNA cleavage was observed in a guide-dependent and RNP concentration-dependent manner. Purified RNP complexes containing randomized gRNA (non-targeting) did not display cleavage activity, whereas targeted sgRNA RNP complexes retained cleavage activity. RNPs having targeted sgRNA displayed complete cleavage was observed at 1.25 uM RNP and 0.25 uM DNA substrate. These results indicate that the co-purified CPP-Cas9 RNPs, as prepared using the plasmid library herein, are catalytically active.

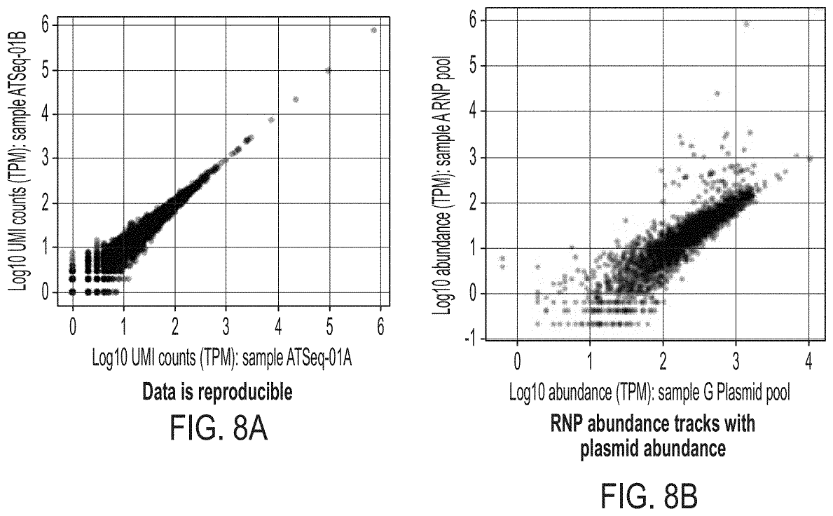

Finally, RNAs that co-purified with the pool of CPP-Cas9 RNPs were analyzed by RNA-seq. FIGS. 8A and 8B graphically depicts results comparing inter-replicate RNA-seq UMI counts (FIG. 8A), showing that the data is reproducible, and sample correlation for plasmid vs RNP abundance (FIG. 8B), showing that RNP abundance tracks with plasmid abundance.

In summary, this example demonstrates that the RNP purification of pool CPP-Cas9 RNPs works, the RNPs successfully co-elute with sgRNA (e.g., uiRNA), and provide a catalytically active CPP-Cas9 RNP that can target cognate dsDNA in vitro.

### Example 4—Co-Incubation of Candidate CPP-Cas9 RNPs with T Cells

The pooled CPP-Cas9 RNPs were assessed for cellular targeting by co-incubating the pooled RNPs with human or mouse T cells. Following co-incubation, nuclear fractionation was performed on the target cells, and RNA was isolated and sequenced from the nuclear extractions. The uiRNAs identified in the isolated nuclear RNAs were used to identify candidate CPPs that effectively facilitated cellular uptake of Cas9.

Pooled CPP-Cas9 RNPs were co-incubated with human T cells (PBMCs, stimulated) or mouse T cells (spleen, stimulated). 2.5 μm of pooled Cas9 RNP was mixed with approximately 200 cells/μl and media. Samples were assessed after 1 hour or 5 hours of incubation with the CPP-Cas9 RNPS (see Table 1 for summary of study design). Negative control samples were co-incubated with buffer but no Cas9 RNP for 5 hours. Cells were immediately lysed and the nuclei and cytoplasm were fractionated from samples obtained at each time point. To separate the nuclear and cytoplasmic fractions, cells were pelleted at 300 RCF for 5 minutes. The supernatant was carefully removed. 200 ul lysis buffer (10 mM tris-Cl, 10 mM NaCl, 3 mM MgCl₂, 0.1% NP-40) were added to resuspended cells and incubated on ice for 5 minutes. The sample was centrifuged at 500 RCF for 5 minutes at 4° C. The supernatant, corresponding to the cytoplasmic fraction, was removed and saved. 1 mL nuclear wash buffer (1×PBS, 1% BSA) was added and the previous steps were repeated twice. A cell strainer was used to remove clumps. Finally, the nuclear fraction was resuspended in the nuclear wash buffer.

TABLE 1
Study Design

| Sample | Condition | Time | Species | Fraction | Replicate |
|---|---|---|---|---|---|
| A | sgRNA pool phenol | | | | 1 |
| | extracted | | | | |
| B | sgRNA pool phenol | | | | 2 |
| | extracted | | | | |
| C | stim-T cells + RNP pool | 1 hr | human | nuc | 1 |
| D | stim-T cells + RNP pool | 1 hr | human | nuc | 2 |
| E | stim-T cells + RNP pool | 5 hr | human | nuc | 1 |
| F | stim-T cells + RNP pool | 5 hr | Human | nuc | 2 |
| G | plasmid pool | | | | 1 |
| H | plasmid pool | | | | 2 |

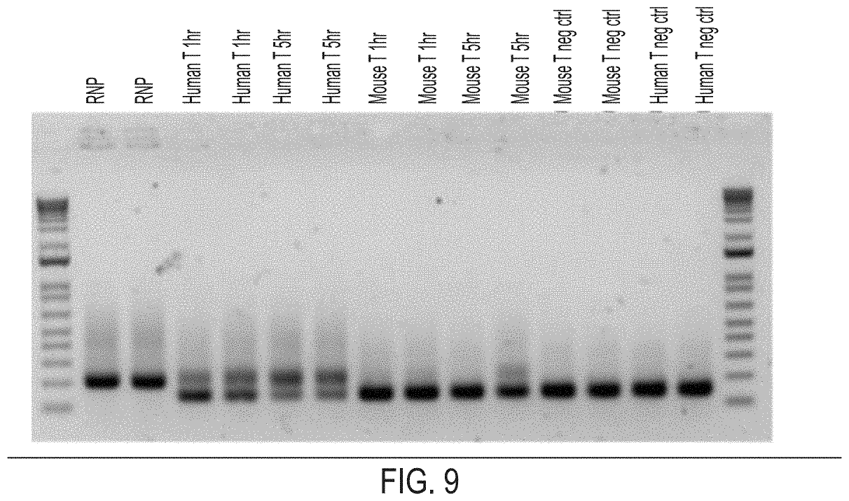

As shown in the upper bands in FIG. 9, products evident of nuclear gRNAs were observed in the nuclear fraction of human T cells after 1 hour and 5 hours of incubating the RNPs with the cells. Further, the amount of barcoded gRNA in the nuclear fraction increased in a time-dependent manner (FIG. 9).

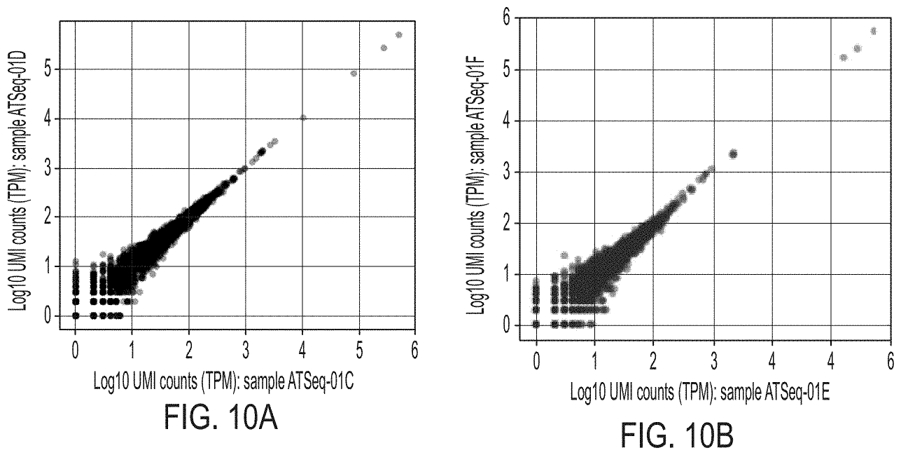

The isolated RNAs were then amplified by reverse transcription (RT) PCR to generate, cDNA products for sequencing. To sequence the cDNA library prepared from isolated nuclear gRNAs, the library of cDNAs were amplified using NEBNext barcoded primer, size-selected by agarose gel, ligated into a plasmid containing a UMI, quantified (QuBit, fragment analyzer), mixed, and sequenced by Illumina sequencing. Based on UMIs count, the RNA-seq inter-replicate UMI counts were consistent between runs for sequencing of RNA isolated from stimulated human T cells incubated with the purified RNP library for 1 hour (FIG. 10A) or 5 hours (FIG. 10B).

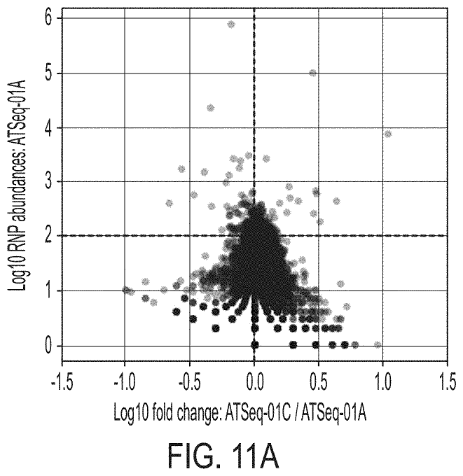

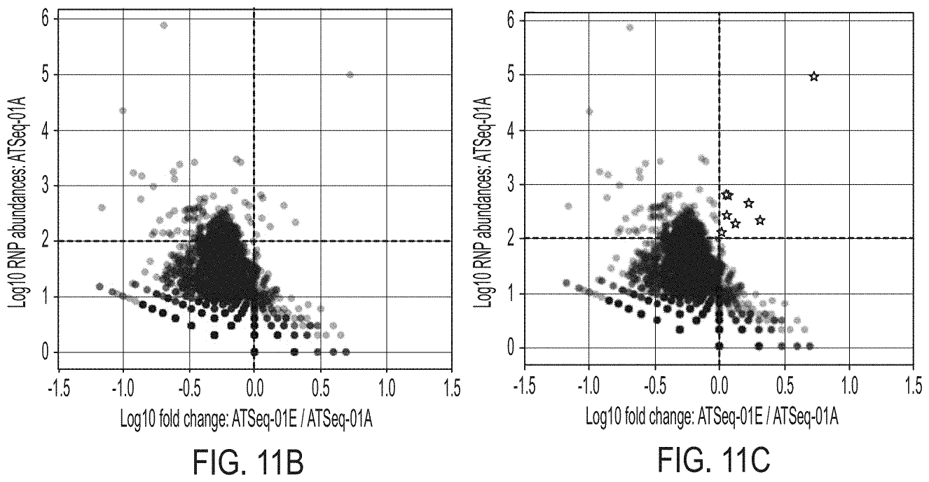

Next, RNAs isolated from human T cells, as outlined above, were analyzed to identify differentially internalized CPP-Cas9 RNPs. The fold change of RNAs sequenced in nuclear extractions (ATSeq-01C) from human stimulated T cells relative to RNAs sequenced in the starting maternal (pooled RNPs prior to co-incubation; ATSeq-01A) was plotted relative to total RNP abundance (ATSeq-01A; y-axis), as shown in FIGS. 11B and 11C. FIG. 11C highlights key data points (starred data points) representing RNAs associated with CPP-Cas9 RNPs having high nuclear internalization in human stimulated T cells following 5 hours of co-incubation with the pool of CPP-Cas9 RNPs. CPPs associated with the highlighted data points are summarized in Table 2.

TABLE 2
Candidate CPPs
Peptide Sequence

IRRGISRK (SEQ ID NO: 1)
KRKRAV (SEQ ID NO: 2)
PKPKRQTKR (SEQ ID NO: 3)
RRRRHCNR (SEQ ID NO: 4)
IPDPTGQS (SEQ ID NO: 5)
IKREREND (SEQ ID NO: 6)
KKLQEQEKQQKVEFRKR (SEQ ID NO: 7)
GPNKKKRKL (SEQ ID NO: 8)
RRRRASAPISQWSSSRRSR (SEQ ID NO: 9)
KLALKLALKALKAALKLA (SEQ ID NO: 10)
FLPLIGRVLSGIL (SEQ ID NO: 11)

These results show that the present screening method could successfully identify candidate CPPs that facilitate uptake of Cas9 (i.e., when complexed with Cas9) based on the presence of uiRNAs in target cells.

### Example 5—Measurement of sgRNA Exchange

To assess whether sgRNA are exchanged between CPP-Cas9 fusions during the plasmid construction and RNP purification process, an experiment is performed to measure uiRNA exchange. Two plasmids are prepared. A first plasmid is prepared that encodes a FLAG affinity-tagged Cas9 protein along with a known sgRNA (i.e., sgRNA-GFP). A second plasmid is prepared that encodes a non-tagged Cas9, along with a randomized uiRNA. These plasmids are mixed together at equal ratios. A pooled bacterial transformation is performed, and RNPs are purified. RNA-seq is performed before and after FLAG affinity pulldown. The abundance of uiRNA or sgRNA-GFP in the RNP pool and the abundance of uiRNA or sgRNA-GFP in the FLAG purified material are assessed. By looking at the ratio of sgRNA-GFP:uiRNA counts in the input material, and in the FLAG pulldown, the frequency of sgRNA exchange is determined. If there is a low-degree of sgRNA exchange, FLAG pulldown material will contain primarily the known GFP sgRNA. In contrast, if a high-degree of exchange occurs, uiRNA counts in the input will correlate with that in the FLAG pulldown material.

### Example 6—Optimization of Library Purification

As described in Example 3, to purify the library of CPP-Cas9, RNPs were purified from E. coli cell lysates using immobilized metal affinity chromatography (IMAC, e.g., using a nickel resin with affinity for a His tag) followed by size exclusion chromatography using an S200 column. However, during purification, DNA contamination was present in the purified RNPs. Further, the Cas9 RNPs in the library partitioned into two major species, indicating the presence of protein or nucleic acid contaminants. To address these issues, an optimized RNP purification protocol was developed.

To remove DNA contaminants, DNase treatment of RNPs was assessed. DNase treatment of RNPs for 1 hour at 30C yielded pure RNPs with reduced DNA contamination. Accordingly, a DNase treatment step was added after the IMAC step.

To remove contaminating proteins and nucleic acids, RNP purity was assessed following both DNase I treatment and anion exchange chromatography. RNPs were treated with DNase I after the IMAC step and before performing anion exchange chromatography. For the anion exchange chromatography, RNPs were applied to a 5 mL HiScreen Q HP column. Although RNP yield was reduced by the addition of the anion exchange chromatography step, anion exchange removed major contaminating proteins and nucleic acids.

Accordingly, the improved RNP purification protocol incorporated DNase treatment and anion exchange chromatography steps, after the IMAC step and prior to the SEC step, to obtain high purity RNPs.

### Example 7—Subcellular Fractionation Validation

To further validate the subcellular fractionation protocol, a quality control experiment was performed using quantitative PCR (qPCR) to track reporter transcripts with known subcellular to evaluate the fractionation workflow. SNROD22 (small nucleolar RNA) was used a control RNA that is known to localize in the nucleus and GAPDH mRNA was used as a control mRNA that is known to localize in the cytosol. Cells were evaluated for the presence of the marker transcripts using qPCR. Consistent with the subcellular localization patterns of each control RNA, nuclear fractions were enriched for SNORD22, and cytoplasmic fractions were enriched for GAPDH.

Next, experiments were performed to assess the correlation between nuclear internalization and genome editing. Cas9 RNPs were generated using a CD47-targeting guide RNA. T cells were co-incubated with RNPs including Cas9, Cas9-2×NLS, or 4×NLS-Cas9-2×NLS, or a no RNP control. Cells were then assessed using either (i) FACS to detect CD47 depletion (a phenotypic readout of genome editing) or (ii) qPCR to detect sgRNA in nuclear fractions obtained from the cells. Nuclear internalization of the sgRNA correlated with genome editing as detected by FACS, indicating that nuclear internalization can serve as a proxy for genome editing.

### Example 8—Optimization of RNA-Seq Library Preparation

The uiRNA utilized in the present examples includes from 5′ to 3′, a barcode to analyze uiRNA using RNA-seq. An alternative method was developed to provide a primer with sequencing handle and a second PCR handle to enable amplification. As described in Example 3, template-switch reverse transcription (e.g., as derived from SMART-seq, 10× Genomics) is one method by which the uiRNA-seq library can be prepared. Preparation of uiRNA-seq library by template-switch reverse transcription, such as by using SMART-seq, is also described in, for example, Picelli et al. (Nat Protoc 9:171-181 (2014)), which is incorporated herein by reference in its entirety. As an alternative method, splinted ligation using T4 DNA ligase was used to prepare a uiRNA-seq library using the SRSLY-seq system, as described in, for example, Troll et al. (BMC Genomics 20:1023 (2019)), which is incorporated herein by reference in its entirety. The libraries generated by the template-switch approach was compared to the SRSLY-seq approach. This comparison indicated that SRSLY-seq produced a higher-yield uiRNA-seq library than the template-switch protocol.

### Example 9—CPP-Cas9 Screen in Fibroblasts

Using a methodology similar to that used in Examples 1-4, a screen was performed to rapidly assay a pool of Cas9-fusion proteins including different test cell penetrating peptides (CPP) for CPPs that effectively facilitate internalization of a Cas9 into fibroblasts. A plurality of unique identifying RNAs (uiRNA) including a guide RNA (gRNA) and a library of polynucleotides encoding 5885 different test CPPs were combinatorially assembled into a vector library encoding Cas9. The CPPs were obtained from CPC scientific, CPPsite2, and the NLSDB databases. The CPPs were approximately 15-34 amino acids per peptide, and included tandem repeats of short peptides. The vectors were assembled such that the CPP was operably linked to Cas9. By sequencing the vector, the uiRNA and test CPP on each vector were identified, thereby providing a reference of pairs of associated uiRNA and test CPPs that could be used to identify CPP-Cas9 ribonucleoproteins based on the presence of the uiRNA at later steps.

Upon expression and isolation of CPP-RNPs comprising the uiRNAs, the pooled library of CPP-Cas9 RNPs was screened for RNPs that have enhanced cellular internalization following co-incubation with mouse fibroblasts. Following co-incubation, nuclear fractionation was performed on the target cells, and RNA was isolated and sequenced from the nuclear extractions. The uiRNAs identified in the isolated nuclear RNAs were used to identify candidate CPPs that effectively facilitated cellular uptake of Cas9 in fibroblasts.

Pooled CPP-Cas9 RNPs were purified using DNase treatment and anion exchange chromatography steps, as described in Example 6. Pooled CPP-Cas9 RNPs were co-incubated with fibroblasts via either a low RNP concentration approach or a high RNP concentration approach. In the low RNP concentration approach, pooled CPP-Cas9 RNP was mixed with approximately 300,000 cells and 90 μl media to a final concentration of 2 μM RNPs. In the high RNP concentration approach, pooled CPP-Cas9 RNP was mixed with approximately 300,000 cells and 30 μl media to a final concentration of 5 μM RNPs. In both approaches, fibroblast cells were co-incubated with the pooled RNPs at 37° C. for 60 min, after which the cells were washed and fractionated into nuclei and cytosol for further analysis by RNA-seq, as described in Example 4.

Prior to expressing and isolating the CPP-RNPs, plasmids expressing each CPP-RNP were assessed by plasmid-seq to map the uiRNA barcode-CPP association. Following fractionation of cells co-incubated with CPP-RNPs, uiRNA present in the nucleus vs cytosol were sequenced using the template-switch reverse transcription method. Unfractionated cells co-incubated with the CPP-Cas9 library were assessed as a comparator. Based on the sequenced barcodes on the uiRNA and the previously established map, CPP-RNPs that were in each subcellular fraction were identified. PCR biases in the uiRNA-seq counts were removed (dedpulicate, demultiplex step) and uiNRAs having differential internalization in the nucleus vs cytosol were identified.

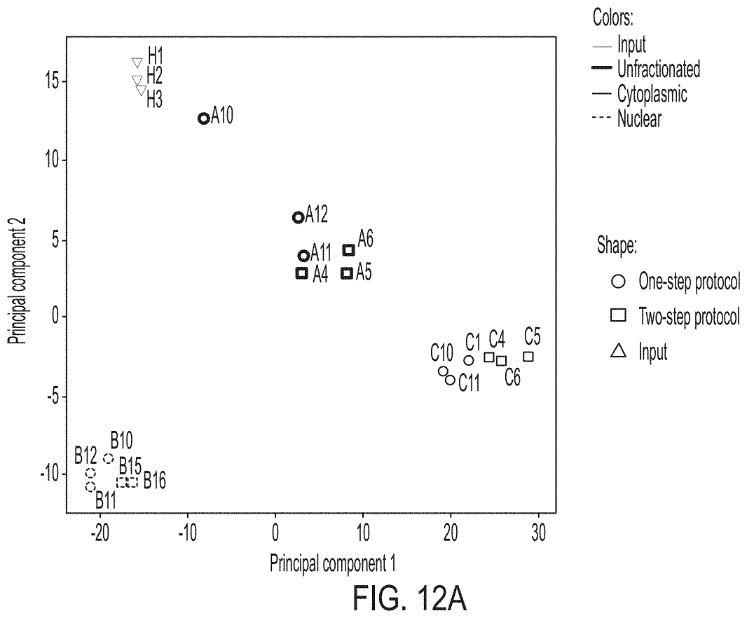

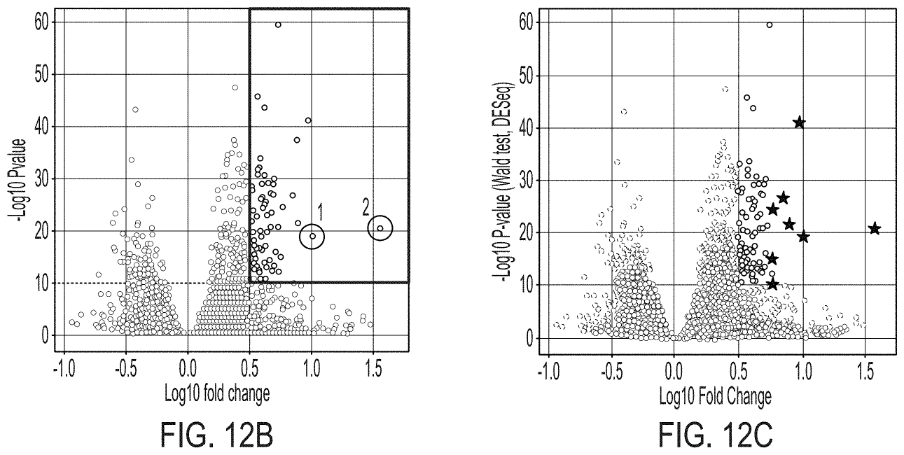

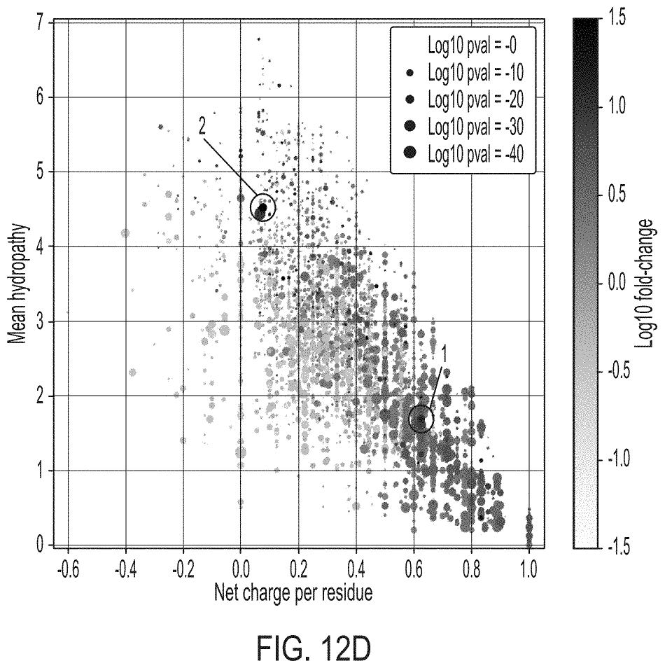

A principal component analysis was performed on the uiRNA counts from an input control (RNAs, such as gRNA, sequenced in the starting material (pooled RNPs prior to co-incubation)), unfractionated cells, cytoplasmic fraction, and nuclear fraction. The results were further analyzed based on the co-incubation protocol used (low RNP concentration vs high RNP concentration protocol). As shown in FIG. 12A, the RNA-seq data clustered based on the subcellular fraction from which the uiRNAs were isolated. The data did not segregate based on the experimental protocol used for co-incubation.

Next, RNAs isolated from fibroblasts, as outlined above, were analyzed to identify CPP-Cas9 RNPs enriched in the nuclear fraction of the fibroblasts. Peptides enriched in the nuclear fraction were identified based on uiRNA in the nucleus having an log 10 fold change greater than or equal to 0.5 relative to RNAs sequenced in the starting material (pooled RNPs prior to co-incubation, i.e., input control). Peptides that had a statistically significant change in abundance in the nucleus relative to the input control were identified based on hits having a Log 10 P-value less than or equal to −10. P-value was computed using the Wald test and Bonferoni corrected for multiple hypothesis testing. A total of 96 hits were identified that were enriched in the nucleus and displayed a significant change in abundance relative to the input control.

The fold change of RNAs in nuclear extractions relative to RNAs in an input control were was plotted in a volcano plot relative to P value (y-axis), as shown in FIG. 12B. RNAs associated with CPP-Cas9 RNPs displaying nuclear localization in fibroblasts following co-incubation with pooled CPP-Cas9 RNPs are shown in the upper right of portion of the graph (see boxed portion of FIG. 12B and starred hits in FIG. 12C).

The identified peptides were further partitioned by chemical property (to determine if CPPs associated with CPP-Cas9 RNP nuclear localization have similar chemical properties. As shown in FIG. 12D, the identified CPPs were partitioned based on hydropathy (y-axis) and net charge per residue (x-axis). Each dot represents a peptide in FIGS. 12B and 12C, wherein the size of the dot indicates the P value (Log 10), and the shading indicates fold change (Log 10). The data on the bottom right of the graph indicate highly charged CPPs with a low degree of hydrophobicity. CPPs associated with enriched nuclear localization and higher P-values were generally highly charged (e.g., greater than +0.4 net charge per residue) and have reduced hydrophobicity, although certain non-polar peptides (see circled data point 2 in FIG. 12D) were also identified that were associated with enriched nuclear localization of CPP-Cas9 RNPs.

FIG. 12C highlights the top eight data points (see starred data points) representing RNAs associated with CPP-Cas9 RNPs having enriched nuclear internalization in fibroblasts. CPPs associated with the highlighted data points are summarized in Table 3. In certain instances, several CPPs were identified with similar sequences. For example, two CPPs were identified in the screen that include the amino acid motif CVQWSLLRGYQPC (SEQ ID NO: 20; see Hits #1 and #3 in Table 3). Further, three CPPs were identified in the screen that include the amino acid motif XKXRX-GSGS (SEQ ID NO: 21), where X is either the amino acid R or K. A majority of hits were polycationic (e.g., R/K-rich). 10 variants of the TAT cell penetrating peptide were additionally identified along with two variants of S41 peptide. Additionally, Bacillus thuringiensis endotoxin delta and penetratin were identified in the screen.

TABLE 3
CPPs associated with uiRNAs (of CPP-Cas9 RNPs) enriched in nuclear
fraction of fibroblasts

| Hit | | Log10 fold | −Log10 |
|---|---|---|---|
| No. | Peptide | change | pvalue |
| 1 | CVQWSLLRGYQPCCVQWSLLRGYQPC | 1.56 | 20.74 |
| | (SEQ ID NO: 12) | | |
| 2 | RKKRKGSGSRKKRKGSGSRKKRKGSGSRKKRK | 1.01 | 19.03 |
| | (SEQ ID NO: 13) | | |
| 3 | CVQWSLLRGYQPCGSGSCVQWSLLRGYQPC | 0.97 | 41.15 |
| | (SEQ ID NO: 14) | | |
| 4 | KKRRRGSGSKKRRRGSGSKKRRRGSGSKKRRR | 0.89 | 21.63 |
| | (SEQ ID NO: 15) | | |
| 5 | RKKRRGSGSRKKRRGSGSRKKRRGSGSRKKRR | 0.85 | 26.84 |
| | (SEQ ID NO: 16) | | |
| 6 | RQIKIWFQNRRMKWKKC | 0.77 | 10.19 |
| | (SEQ ID NO: 17) | | |
| 7 | GRKRKRSGRKRKRSGRKRKRSGRKRKRS | 0.77 | 24.57 |
| | (SEQ ID NO: 18) | | |
| 8 | PRKKRGRPRKKRGRPRKKRGRPRKKRGR | 0.76 | 15.08 |
| | (SEQ ID NO: 19) | | |

Sequence Table

| SEQ ID | | |
|---|---|---|
| NO | Description | Sequence |
| 1 | CPP1 | IRRGISRK |
| | (amino acid sequence) | |
| 2 | CPP2 | KRKRAV |
| | (amino acid sequence) | |
| 3 | CPP3 | PKPKRQTKR |
| | (amino acid sequence) | |
| 4 | CPP4 | RRRRHCNR |
| | (amino acid sequence) | |
| 5 | CPP5 | IPDPTGQS |
| | (amino acid sequence) | |
| 6 | CPP6 | IKREREND |
| | (amino acid sequence) | |
| 7 | CPP7 | KKLQEQEKQQKVEFRKR |
| | (amino acid sequence) | |
| 8 | CPP8 | GPNKKKRKL |
| | (amino acid sequence) | |
| 9 | CPP9 | RRRRASAPISQWSSSRRSR |
| | (amino acid sequence) | |
| 10 | CPP10 | KLALKLALKALKAALKLA |
| | (amino acid sequence) | |
| 11 | CPP11 | FLPLIGRVLSGIL |
| | (amino acid sequence) | |
| 12 | CPP12 | CVQWSLLRGYQPCCVQWSLLRGYQPC |
| | (amino acid sequence) | |
| 13 | CPP13 | RKKRKGSGSRKKRKGSGSRKKRKGSGSRKKRK |
| | (amino acid sequence) | |
| 14 | CPP14 | CVQWSLLRGYQPCGSGSCVQWSLLRGYQPC |
| | (amino acid sequence) | |
| 15 | CPP 15 | KKRRRGSGSKKRRRGSGSKKRRRGSGSKKRRR |
| | (amino acid sequence) | |
| 16 | CPP16 | RKKRRGSGSRKKRRGSGSRKKRRGSGSRKKRR |
| | (amino acid sequence) | |
| 17 | CPP17 - Penetratin | RQIKIWFQNRRMKWKKC |
| | Cys | |
| 18 | [GRKRKRS]₄ | GRKRKRSGRKRKRSGRKRKRSGRKRKRS |
| 19 | [PRKKRGR]₄ | PRKKRGRPRKKRGRPRKKRGRPRKKRGR |
| 20 | CPP Motif 1 | CVQWSLLRGYQPC |
| 21 | CPP Motif 2 | XKXRX-GSGS, where X is either the |
| | | amino acid R or K |
| 22 | 6xHis | HHHHHH |
| 23 | HIV-TAT | RKKRRQRRR |
| 24 | HIV-REV | RQARRNRRRRWR |
| 25 | NLS | PKKKRKV |
| 26 | dodecahistidine | HHHHHHHHHHHH |

What is claimed:

1. A method of identifying a cell targeting agent, the method comprising:
providing a plurality of ribonucleoproteins (RNPs) each comprising an RNA-guided nuclease fusion protein and a unique identifying RNA (uiRNA), wherein the RNA-guided nuclease fusion protein comprises an RNA-guided nuclease, or a functional fragment thereof, and a test protein; and wherein the uiRNA comprises a guide RNA (gRNA) and a sequence identifier;
contacting the RNPs with a population of target cells;
isolating RNA from the population of target cells, thereby obtaining isolated RNA; and
testing the isolated RNA for the presence of the identifier sequence, wherein the presence of the identifier sequence indicates that the test protein is a cell targeting agent.

2. A method of identifying a cell targeting agent, the method comprising:
providing a vector encoding an RNA-guided nuclease fusion protein comprising an RNA-guided nuclease, or a functional fragment thereof, and a test protein, and encoding a unique identifying RNA (uiRNA) comprising a guide RNA (gRNA) and a sequence identifier;
transferring the vector to a host cell suitable to express the RNA-guided nuclease fusion protein and the uiRNA;
expressing the RNA-guided nuclease fusion protein and the uiRNA in the host cell, such that ribonucleoproteins (RNPs) each comprising the RNA-guided nuclease fusion protein and the uiRNA are formed;
isolating the RNPs from the host cell;
contacting the RNPs with a population of target cells;
isolating RNA from the population of target cells; and
testing the isolated RNA for the presence of the identifier sequence, wherein the presence of the identifier sequence indicates that the test protein is a cell targeting agent.

3. The method of claim 2, wherein portions of the vector encoding the nucleic acid sequence identifier and the test protein are sequenced prior to the vector being transferred into the host cell, thereby providing a reference for identifying the test protein.

4. The method of any one of claims 1-3, wherein the presence of the identifier sequence is detected using polymerase chain reaction (PCR) or a nucleic acid microarray.

5. The method of any one of claims 2-4, wherein the vector is in a plurality of vectors and the plurality of vectors are transferred into host cells under conditions such that the average vector per host cell is 1 or more.

6. The method of any one of claims 2-5, wherein the vector comprises a first promoter operatively linked to a nucleic acid sequence encoding the RNA-guided nuclease fusion protein, and comprises a second promoter operatively linked to a nucleic acid sequence encoding the uiRNA.

7. The method of claim 6, wherein the first and second promoter are each inducible such that the expression level of the RNA-guided nuclease fusion protein and the expression level of the uiRNA can be controlled to obtain RNPs.

8. The method of claim 6, wherein the first and/or second promoter is a constitutive promoter.

9. The method of any one of claims 1-8, wherein the vector comprises a selectable marker to select for the host cell into which the vector has been transferred.

10. The method of any one of claims 2-9, wherein the vector comprises a bacterial origin of replication.

11. The method of any one of claims 2-9, wherein the vector comprises a eukaryotic origin of replication.

12. The method of any one of claims 1-11, wherein the cell targeting agent either internalizes into a compartment of the target cell or binds to the cell surface of the target cell.

13. The method of claim 12, wherein the compartment is a membrane-bound organelle or cytoplasm.

14. The method of claim 13, wherein the membrane-bound organelle is a nucleus, endoplasmic reticulum, Golgi apparatus, vacuole, lysosome, endosome, or mitochondria.

15. The method of any one of claims 1-14, wherein the isolated RNA is obtained from membrane-bound organelles that are extracted from the target cell prior to RNA isolation.

16. The method of any one of claims 1-13, wherein the isolated RNA is obtained from cytoplasm that is extracted from the target cell prior to RNA isolation.

17. The method of any one of claims 1-16, wherein the testing step comprises reverse-transcribing the isolated RNA to producing cDNA, and sequencing the cDNA to determine the presence of the identifier sequence.

18. The method of any one of claims 1-16, wherein the testing step comprises sequencing the isolated RNA to determine the presence of the identifier sequence.

19. The method of any one of claims 1-18, wherein the test protein is a peptide.

20. The method of any one of claims 1-18, wherein the test protein is an antigen-binding protein.

21. The method of claim 20, wherein the antigen binding protein is a nanobody, a domain antibody, an scFv, a Fab, a diabody, a BiTE, a diabody, a DART, a minibody, a F(ab′)₂, an intrabody, or an antibody mimetic.

22. The method of claim 21, wherein the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, or a versabody, an aptamer, or a cyclotide.

23. The method of any one of claims 1-17, wherein the test protein is a ligand, or portion thereof.

24. The method of any one of claims 1-23, wherein the host cell is a eukaryotic cell.

25. The method of any one of claims 1-23, wherein the host cell is a bacterial cell.

26. The method of claim 25, wherein the bacterial cell is E. coli.

27. The method of any one of claims 1-26, wherein the RNA-guided nuclease is a Class 2 Cas polypeptide.

28. The method of claim 27, wherein the Class 2 Cas polypeptide is a Type II, Type V, or Type VI Cas polypeptide.

29. The method of claim 28, wherein the Type II Cas polypeptide is Cas9.

30. The method of any one of claims 1-29, wherein the target cells are mammalian cells.

31. The method of claim 30, wherein the mammalian cells are hematopoietic stem cells (HSC), neutrophils, T cells, B cells, dendritic cells, macrophages, ocular cells, or fibroblasts.

32. A cell expression vector comprising:
a nucleic acid encoding an RNA-guided nuclease operably linked to a cloning site for inserting a nucleic acid of a test protein, thereby forming an RNA-guided nuclease fusion protein comprising the RNA-guided nuclease and the test protein; and
a nucleic acid encoding a unique identifying RNA (uiRNA), wherein the uiRNA comprises a guide RNA and a sequence identifier.

33. The cell expression vector of claim 32, further comprising the nucleic acid encoding the test protein.

34. The cell expression vector of claim 32 or 33, wherein the expression vector is a plasmid.

35. The cell expression vector of any one of claims 32-34, wherein the cell expression vector comprises a first promoter operatively linked to the nucleic acid sequence encoding the RNA-guided nuclease, and comprises a second promoter operatively linked to the nucleic acid sequence encoding the uiRNA.

36. The cell expression vector of claim 35, wherein the first and second promoter each comprise an inducible element such that the expression level of the RNA-guided nuclease fusion protein and the expression level of the uiRNA can be controlled.

37. The cell expression vector of claim 35 or 36, wherein the first and/or second promoter is T7 or T5.

38. The cell expression vector of claim 35, wherein the first and/or second promoter is a constitutive promoter.

39. The cell expression vector of any one of claims 32-38, wherein the vector comprises a selectable marker.

40. The cell expression vector of any one of claims 32-39, wherein the vector comprises a bacterial origin of replication.

41. The cell expression vector of any one of claims 32-39, wherein the vector comprises a eukaryotic origin of replication.

42. The cell expression vector of any one of claims 32-41, wherein the RNA-guided nuclease is a Class 2 Cas polypeptide.

43. The cell expression vector of claim 42, wherein the Class 2 Cas polypeptide is a Type II, Type V, or Type VI Cas polypeptide.

44. The cell expression vector of claim 43, wherein the Type II Cas polypeptide is Cas9.

45. A kit comprising the cell expression vector of any one of claims 32-44.

46. The kit of claim 45, wherein the kit further comprises reagents for inserting the polynucleotide encoding the test protein into the cloning site of the cell expression vector.

47. An isolated cell comprising the cell expression vector of any one of claims 32-44.

48. The cell of claim 47, wherein the cell is a eukaryotic cell or a bacterial cell.

49. The cell of claim 48, wherein the eukaryotic cell is a mammalian cell, a yeast cell, or an insect cell.

50. The cell of claim 49, wherein the mammalian cell is a COP cell, an L cell, a C127 cell, an Sp2/0 cell, an NS-0 cell, an NIH3T3 cell, a PC12 cell, a PC12h cell, a BHK cell, a CHO cell, a COS1 cell, a COS3 cell, a COST cell, a CV1 cell, a Vero cell, a HeLa cell, an HEK-293 cell, a PER C6 cell, a cell derived from diploid fibroblasts, a myeloma cell, or HepG2.

51. The cell of claim 49, wherein the yeast cell is Pichia pastoris or Saccharomyces cerevisiae and the insect cell is Spodoptera frugiperda.

52. The cell of claim 48, wherein the bacterial cell is an E. coli cell.

53. A method for producing at least one RNP comprising the RNA-guided nuclease fusion protein and the uiRNA comprising culturing a cell comprising the expression vector of any one of claims 32-44 in a cell culture medium under conditions allowing expression and assembly of the at least one RNP.

54. The method of claim 51, wherein the at least one RNP is/are secreted into the cell culture medium and the method further comprises the step of isolating from the cell culture medium the at least one RNP.

55. A library of cell expression vectors comprising a plurality of the cell expression vector of any one of claims 32-44.

56. The library of claim 55, wherein each of the cell expression vectors comprises a different sequence identifier.


SEQUENCE LISTING (26 sequences)

SEQ ID NO: 1 - protein, 8 aa, Artificial Sequence
  source: /note=“Description of Artificial Sequence Synthetic peptide”
IRRGISRK

SEQ ID NO: 2 - protein, 6 aa, Artificial Sequence
  source: /note=“Description of Artificial Sequence Synthetic peptide”
KRKRAV

SEQ ID NO: 3 - protein, 9 aa, Artificial Sequence
  source: /note=“Description of Artificial Sequence Synthetic peptide”
PKPKRQTKR

SEQ ID NO: 4 - protein, 8 aa, Artificial Sequence
  source: /note=“Description of Artificial Sequence Synthetic peptide”
RRRRHCNR

SEQ ID NO: 5 - protein, 8 aa, Artificial Sequence
  source: /note=“Description of Artificial Sequence Synthetic peptide”
IPDPTGQS

SEQ ID NO: 6 - protein, 8 aa, Artificial Sequence
  source: /note=“Description of Artificial Sequence Synthetic peptide”
IKREREND

SEQ ID NO: 7 - protein, 17 aa, Artificial Sequence
  source: /note=“Description of Artificial Sequence Synthetic peptide”
KKLQEQEKQQKVEFRKR

SEQ ID NO: 8 - protein, 9 aa, Artificial Sequence
  source: /note=“Description of Artificial Sequence Synthetic peptide”
GPNKKKRKL

SEQ ID NO: 9 - protein, 19 aa, Artificial Sequence
  source: /note=“Description of Artificial Sequence Synthetic peptide”
RRRRASAPISQWSSSRRSR

SEQ ID NO: 10 - protein, 18 aa, Artificial Sequence
  source: /note=“Description of Artificial Sequence Synthetic peptide”
KLALKLALKALKAALKLA

SEQ ID NO: 11 - protein, 13 aa, Artificial Sequence
  source: /note=“Description of Artificial Sequence Synthetic peptide”
FLPLIGRVLSGIL

SEQ ID NO: 12 - protein, 26 aa, Artificial Sequence
  source: /note=“Description of Artificial Sequence Synthetic peptide”
CVQWSLLRGYQPCCVQWSLLRGYQPC

SEQ ID NO: 13 - protein, 32 aa, Artificial Sequence
  source: /note=“Description of Artificial Sequence Synthetic polypeptide”
RKKRKGSGSRKKRKGSGSRKKRKGSGSRKKRK

SEQ ID NO: 14 - protein, 30 aa, Artificial Sequence
  source: /note=“Description of Artificial Sequence Synthetic polypeptide”
CVQWSLLRGYQPCGSGSCVQWSLLRGYQPC

SEQ ID NO: 15 - protein, 32 aa, Artificial Sequence
  source: /note=“Description of Artificial Sequence Synthetic polypeptide”
KKRRRGSGSKKRRRGSGSKKRRRGSGSKKRRR

SEQ ID NO: 16 - protein, 32 aa, Artificial Sequence
  source: /note=“Description of Artificial Sequence Synthetic polypeptide”
RKKRRGSGSRKKRRGSGSRKKRRGSGSRKKRR

SEQ ID NO: 17 - protein, 19 aa, Artificial Sequence
  source: /note=“Description of Artificial Sequence Synthetic peptide”
RQIKIWFQNRRMKWKKGGC

SEQ ID NO: 18 - protein, 28 aa, Artificial Sequence
  source: /note=“Description of Artificial Sequence Synthetic peptide”
GRKRKRSGRKRKRSGRKRKRSGRKRKRS

SEQ ID NO: 19 - protein, 28 aa, Artificial Sequence
  source: /note=“Description of Artificial Sequence Synthetic peptide”
PRKKRGRPRKKRGRPRKKRGRPRKKRGR

SEQ ID NO: 20 - protein, 13 aa, Artificial Sequence
  source: /note=“Description of Artificial Sequence Synthetic peptide”
CVQWSLLRGYQPC

SEQ ID NO: 21 - protein, 9 aa, Artificial Sequence
  source: /note=“Description of Artificial Sequence Synthetic peptide”
  VARIANT (1)..(1): /replace=“Lys”
  VARIANT (3)..(3): /replace=“Lys”
  VARIANT (5)..(5): /replace=“Lys”
  SITE (1)..(9): /note=“Variant residues given in the sequence have no preference with respect to those in the annotations for variant positions”
RKRRRGSGS

SEQ ID NO: 22 - protein, 6 aa, Artificial Sequence
  source: /note=“Description of Artificial Sequence Synthetic 6xHis tag”
HHHHHH

SEQ ID NO: 23 - protein, 9 aa, Human immunodeficiency virus 1
RKKRRQRRR

SEQ ID NO: 24 - protein, 12 aa, Human immunodeficiency virus 1
RQARRNRRRRWR

SEQ ID NO: 25 - protein, 7 aa, Simian virus 40
PKKKRKV

SEQ ID NO: 26 - protein, 12 aa, Artificial Sequence
  source: /note=“Description of Artificial Sequence Synthetic peptide”
HHHHHHHHHHHH